(19) United States
(12) Patent Application Publication
(10) Pub. No.: US 2025/0110033 A1
(43) Pub. Date: Apr. 3, 2025

(54) SYSTEMS AND METHODS TO EVALUATE A FOAMER FOR UNLOADING LIQUID IN OIL AND GAS WELLS OF MATURE FIELDS

(71) Applicant: Weatherford Technology Holdings, LLC, Houston, TX (US)

(72) Inventors: John E. Busteed, Houston, TX (US); Mohammed Omer, Hyderabad (IN); Francisco Eugenio Fragachan, Katy, TX (US)

(21) Appl. No.: 18/513,476

(22) Filed: Nov. 17, 2023

Related U.S. Application Data
(60) Provisional application No. 63/586,994, filed on Sep. 29, 2023.

(51) Int. Cl. G01N 7/16; E21B 43/16; G01N 33/00

(52) U.S. Cl. CPC G01N 7/16; E21B 43/168; G01N 33/0067

(57) ABSTRACT

Systems and methods are provided for evaluating a foamer for use in an oil and gas well for unloading of a liquid. The systems and methods provide for methods including: (a) combining (i) an aqueous phase, a hydrocarbon phase, or both an aqueous phase and a hydrocarbon phase in a predetermined proportion with (ii) a foamer to obtain a liquid, wherein the foamer is in a predetermined concentration in the liquid; (b) sparging the liquid with a gas at a predetermined gas flow rate to create a foam from at least some of the liquid and at least some of the gas; and (c) during or after the step of sparging, determining the amount of the liquid in the foam, wherein the step of determining is performed one or more times.

### CROSS-REFERENCE TO RELATED APPLICATIONS

This application claims the priority to U.S. provisional application No. 63/586,994 filed Sep. 29, 2023, entitled Systems and Methods to Evaluate a Foamer for Unloading Liquid in Oil and Gas Wells or Mature Fields, and having for named inventors John E. Busteed, Mohammed Omer, and Francisco Eugenio Fragachan, which is incorporated herein by reference.

### TECHNICAL FIELD

The disclosure is in the field of laboratory small-scale testing for evaluating the selection and performance of a foamer surfactant in a fluid system and environment simulating a real fluid system and real conditions in an oil and gas well, and, more particularly, regarding the unloading of liquid in mature fields.

### BACKGROUND

In oil and gas wells of mature fields where the reservoirs are depleting, liquid loading has become one of the common problems. Liquid loading problems occur when gas flow rates are not sufficient to lift associated reservoir liquid from downhole in the well to the wellhead at the surface.

During upward fluid flow in a vertical tubular in a well, a gas tends to move upward faster than a liquid. This is primarily due to density differences between the gas and liquid in a well, although other characteristics of the fluids can have an effect. Depending on the different phase velocities, different flow patterns are formed in tubulars. As gas velocities range from closer to the lower velocity of the liquid to higher velocities relative to the velocity of the liquid, the basic flow patterns tend to progressively range from bubble, to slug, to churn, to annular-mist flow patterns.

FIGS. 1A-1D are illustrations of two-phase flow patterns between a liquid phase and a gas phase moving upward through a vertical tubular 10 in a well (not shown), where FIG. 1A illustrates an annular-mist flow pattern, FIG. 1B illustrates a churn flow pattern, FIG. 1C illustrates a slug flow pattern, and FIG. 1D illustrates a bubble flow pattern.

Referring to FIG. 1A, when the gas velocity is higher relative to the velocity of the liquid such that the flow pattern in the vertical tubular 10 is an annular-mist flow pattern, there is a vortex of a gas core 14 flowing upward along the axial center of the vertical tubular 10 and pushing the liquid to a relatively thin film or layer 12 circumferentially along the inner wall of the vertical tubular 10. At the gas-liquid interface between the gas core 14 and the liquid layer 12 along the inner wall of the vertical tubular 10, the shear between the different fluids causes droplets 16 of the liquid to be carried into and suspended as a mist in the gas core 14. In addition, in the annular-mist flow pattern, some of the droplets 16 are deposited back into the liquid film or layer 12. This is a continuous interplay between the gas phase of the gas core 14 and the liquid phase of the liquid layer 12 in the annular-mist flow pattern.

Referring to FIGS. 1B, 1C, and 1D, at lower upward gas velocities relative to the upward liquid velocity that are insufficient for the annular-mist flow pattern, the liquid flow is shown bridging at least some of the axial center of the vertical tubular 10, as in the flow patterns of churn, slug, or bubble. The gas velocity in the bubble flow pattern is slower than the slug or churn flow patterns. The churn and slug flow patterns can be unstable.

Referring to FIG. 1B illustrating the churn flow pattern, there is liquid bridging 22 at least some of the inside of the vertical tubular 10 and there is gas churn 24 in and with the liquid bridging 22.

Referring to FIG. 1C illustrating the slug flow pattern, there is liquid bridging 32 entirely across portions of the inside of the vertical tubular 10 and there are gas slugs 34 and some gas bubbles 36 in the liquid bridging 32.

Referring to FIG. 1D illustrating the bubble flow pattern, there is a liquid column 42 substantially completely filling the vertical tubular 10 and there are gas bubbles 44 in the liquid column 42.

The amount of liquid per length of tubular is referred to as “liquid holdup.” Liquid holdup varies depending on the flow pattern. In the range of these flow patterns, as gas velocity relative to the liquid velocity increases, the liquid holdup increases. The liquid holdup is relatively low in an annular-mist flow pattern and relatively high in a bubble flow pattern. Therefore, the annular-mist flow pattern is preferred in gas wells that produce liquids.

Over time, an increasing liquid holdup in the tubulars of a well results in increased hydrostatic pressure on the producing subterranean formation of a well, which left uncorrected can stop the production altogether. The stopping of production due to the hydrostatic pressure of liquid on the formation is referred to as “liquid loading.” (Liquid loading should not be confused with liquid holdup.)

Injection of a surfactant into the well is one of the methods to mitigate increasing liquid holdup and to increase stable production. More particularly, a “foamer,” also known as a “foaming agent” or “foaming surfactant,” is a type of surfactant that helps create a foam of a gas and a liquid. The application of a foamer is a way to improve the efficiency of gas lift by reducing the average weight of the fluid column in the tubing and reducing flow instability resulting from the slug flow pattern and the churn flow pattern.

It is important to be able to evaluate a foamer for its suitability for use in treating a well to reduce liquid loading and increase stable gas production from a well.

### SUMMARY

In an aspect of the disclosure, a system is provided for evaluating a foamer for unloading liquid, the system comprising:
(a) a temperature-controlled bath having a first vessel for containing a liquid in the first vessel at a temperature controlled by the temperature-controlled bath and for measuring the volume of a liquid or foam in the first vessel;
(b) a gas tubing operatively connected from a gas flow meter and a gas source to a gas delivery tubing and a frit for sparging the liquid in the first vessel with a gas for making a foam;
(c) a mass balance;
(d) a second vessel on the mass balance; and
(e) fluid tubing operatively connected from the first vessel in the temperature-controlled bath to the second vessel.

In a further embodiment, the system additionally comprises a condenser for the fluid tubing.

A method for evaluating a foamer is provided, the method comprising the steps of:
(a) combining (i) an aqueous phase, a hydrocarbon phase, or both an aqueous phase and a hydrocarbon phase in a predetermined proportion with (ii) a foamer to obtain a liquid, wherein the foamer is in a predetermined concentration in the liquid;
(b) sparging the liquid with a gas under sparging conditions including a predetermined gas flow rate to create a foam from at least some of the liquid and at least some of the gas; and
(c) during or after the step of sparging, determining the amount of the liquid in the foam, wherein the step of determining is performed one or more times.

In a further embodiment, the step of determining the mass or the amount of the liquid in the foam includes the steps of:
(a) collecting the foam to obtain a collected foam and any of the liquid dropped from the collected foam; and
(b) measuring the mass of the collected foam and any of the liquid dropped from the collected foam.

In yet a further embodiment, the step of collecting the foam additionally comprises cooling the foam to reduce any evaporation of the liquid in the foam to the atmosphere.

Detailed embodiments and examples according to the principles of the principles of the disclosure are provided. However, specific portions or functional details disclosed herein are merely representative for purposes of describing example embodiments. Example embodiments can be embodied in many alternate forms and should not be construed as limited to only the embodiments set forth herein. Example embodiments can have various combinations, modifications, equivalents, and alternatives.

### BRIEF DESCRIPTION OF THE DRAWING

The accompanying figures of the drawing are incorporated into the specification to help illustrate examples according to various embodiments of the disclosure. Like references are used for like elements or features throughout the figures of the drawing. The figures of the drawing are not necessarily to scale.

These figures together with the description explain the general principles of the disclosure. The figures are only for the purpose of illustrating preferred and alternative examples of how the various aspects of the claimed inventions can be made and used and are not to be construed as limiting the claimed inventions to only the illustrated and described examples. Various advantages and features of the various aspects of the present inventions will be apparent from a consideration of the drawing.

FIGS. 1A-1D are illustrations of two-phase flow patterns between a liquid phase and a gas phase moving upward through a vertical tubular 10 in a well (not shown), where FIG. 1A illustrates an annular-mist flow pattern, FIG. 1B illustrates a churn flow pattern, FIG. 1C illustrates a slug flow pattern, and FIG. 1D illustrates a bubble flow pattern.

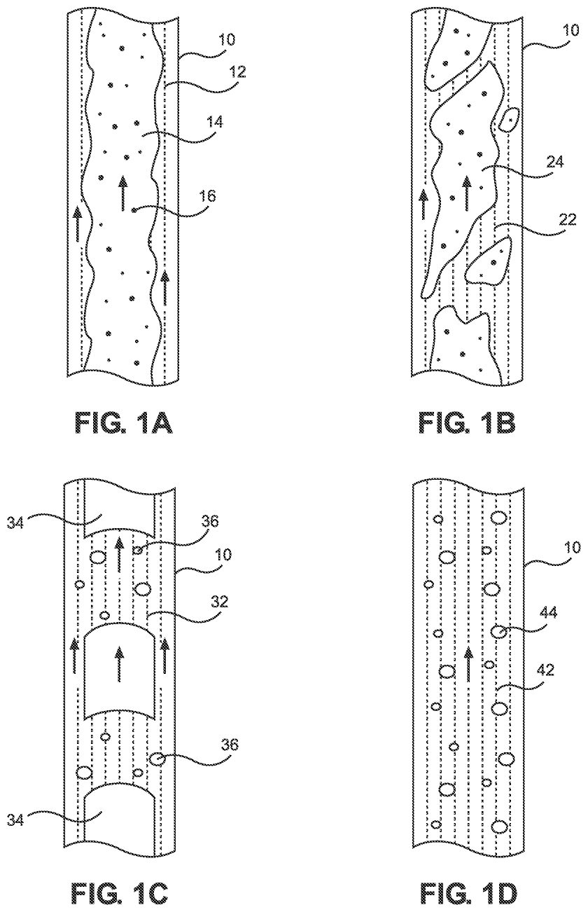

FIG. 2 is an illustration of a conventional (prior art) system 50 of laboratory equipment used for foam testing and evaluation, where the system 50 includes a blender or mixer 52, a graduated cylinder 54, one or more pipettes or syringes 56 (which can be optionally and conveniently held upright on a syringe stand 57), and a stopwatch 58.

FIG. 3 is an illustration of a basic system 100 according to an embodiment of the present disclosure, where the system 100 includes graduated cylinder 104, one or more pipettes or syringes 106 (which can optionally and conveniently be held upright on a syringe stand 107), a stopwatch 108, a laboratory stand 110, a gas source, such as an air a variable rate gas compressor 111, a first gas tubing 112 operatively connecting from the gas compressor 111 to a gas flow meter 114 conveniently supported by the laboratory stand 110, a second gas tubing 115 operatively connecting from the gas flow meter 114 to gas delivery tubing 116 having a frit 120 for sparging a gas down into the graduated cylinder 104.

FIG. 4 is an illustration of an example observation and measurement that can be made in the graduated cylinder 104 of the system 100 of FIG. 3, where after initially foaming of a liquid by sparging with a gas (from the gas compressor 111, not shown in FIG. 4) via the gas delivery tubing 116 down to the frit 120 positioned at or adjacent to the bottom 104b of the graduated cylinder 104, the volume of a liquid column 121 at the bottom of the graduated cylinder 104 can be measured in the graduated cylinder at the liquid level of arrow 123 using the graduated markings 104e, and further where the total volume of the liquid column 121 plus a foam column 125 can be measured in the graduated cylinder 104 at arrow 127 using the graduated markings 104e. The graduated markings 104e can include, for example, volume measurement markings of 1, 2, 3, 4, 5, 6, 7, 8, 9 that are graduated from the bottom 104b of the graduated cylinder 104 upwards in increments of 100 mL (and unlabeled subdivisions in increments of 10 mL) in the case of a graduated cylinder 104 having a total volume of 1,000 mL.

FIG. 5 is an illustration of an embodiment of a system 200 according to the disclosure that includes a temperature-controlled bath apparatus 201, where the temperature-controlled bath apparatus 201 has a temperature-controlled bath 202 of a liquid medium (such as water) that can be heated to a predetermined temperature, a graduated cylinder 204 (preferably like the graduated cylinder 104 illustrated in FIG. 4), where the graduated cylinder 204 can be positioned down into and substantially submerged in the liquid medium of the bath 202, a transparent window 208 (preferably of glass) to see into the temperature-controlled bath 202 and to the graduated cylinder 204, where the graduated cylinder 204 has graduated markings 204e and where the temperature-controlled bath apparatus 201 preferably has a backlight (not shown in FIG. 5) so that the graduated cylinder 204 can be backlit in the temperature-controlled bath 202, a gas flow meter 214, which can be optionally and conveniently built in the temperature-controlled apparatus 201, and a control panel 230 used for controlling the temperature of the temperature-controlled bath 202. In this system 200, the graduated cylinder 204 preferably additionally has a sealable lid 206, a first port 206a through the sealable lid 206, and a second port 206b through the sealable lid 206. In this system 200, a gas source, such as an air compressor 211, is operatively connected by the first gas tubing 212 (going behind and into the apparatus 201) and to the built-in gas flow meter 214 of the temperature-controlled bath apparatus 201, and the gas flow meter 214 is operatively connected by the second gas tubing 215 to the gas delivery tubing 216 that can be positioned to go down through the first port 206a in the lid 206 for the graduated cylinder 204 to position a frit 220 at or near the bottom of the graduated cylinder 204.

FIG. 6 is an illustration of another embodiment of a system according to the disclosure, wherein a system 300 that is like the system 200 illustrated in FIG. 5 (with the same referenced elements) additionally includes a mass balance 340, a second graduated cylinder 342 positioned on the mass balance 340, a laboratory stand 344 to support a third gas tubing 346, where the third gas tubing 346 is operatively connected to the second port 206b of the lid 206 for the graduated cylinder 204. The third gas tubing 346 is for conducting gas or foam from the graduated cylinder 204 and delivering it such that any foam or any liquid dropped from the foam can have the volume measured by the second graduated cylinder 342 or the mass measured by the mass balance 340. (Preferably, as illustrated in FIG. 15, the mass balance 340 and the second graduated cylinder 342 are positioned in a laboratory fume hood 490, where the fume hood 490 is to reduce exposure of laboratory technicians to potentially toxic vapors or gases.)

FIG. 7 is a schematic of a system 400 like the system 300 illustrated in FIG. 6 but additionally showing a camera and computer control, where the system 400 includes a temperature-controlled bath 410 for a first vessel 402, a gas source 411, a gas tubing 412 operatively connected from the gas source 411 through a gas flow meter 414 and from there the gas tubing 412 operatively connected to continue from the gas flow meter 414 to and down into the first vessel 402 for sparging a liquid in the first vessel 402 with a gas from the gas source 411, fluid tubing 415 from the first vessel 402 in the temperature-controlled bath 410 to a second vessel 424 positioned on a mass balance 420, at least one camera 430 for taking pictures or a video recording of the appearance of a liquid or foam in the first vessel 402 and for measuring the volume of a liquid or of a foam vs. time or of the total volume of liquid and foam in the first vessel 402 of the temperature-controlled bath 410 (and optionally one or more other sensors with the camera 430), a central processing unit (“CPU”) 440, where the CPU 440 is operatively connected to obtain and record mass measurements from the mass balance 420, where the CPU 440 is additionally operatively connected to control or receive images or information data from the camera 430 and any optional sensors, and where the CPU 440 has a user interface 450 operatively connected to the CPU 440. The user interface 450 can be, for example, a computer screen or a printer. As any foam is collected from the first vessel 402 into the second vessel 424 and as the foam column collapses in the second vessel 424, the entrained liquid in the foam collects in the second vessel 424 and the gas from the foam can flow out of an upper opening 425 of the second vessel 424.

FIG. 8 is an example of a graph of test data that can be manually recorded with the system 300 illustrated in FIG. 6 or automatically recorded with the system 400 illustrated in FIG. 7, where the graph of FIG. 8 shows the amount of foam or liquid (in grams) collected in the second graduated cylinder 342 on the mass balance 340 of the system 300 illustrated in FIG. 6 or collected in the second vessel 424 on the mass balance 420 of the system 400 illustrated in FIG. 7 (in measurement readings over time, e.g., every 2 to 5 seconds). The mass of the foam is essentially that of the liquid of the foam because the mass of the gas of the foam is negligible.

FIG. 9 is a graphical presentation of Example A of a foamer evaluation according to an embodiment such as the system 200 illustrated in FIG. 5.

FIG. 10 is a graphical presentation of Example B of a foamer evaluation according to an embodiment such as the system 200 illustrated in FIG. 5.

FIG. 11 is a graphical comparison according to Example C of the difference between measurements of the decreasing foam volume (mL) over time vs the liquid volume (mL) dropping out of a foam over time, where FIG. 11 shows data after stopping gas flow.

FIG. 12 is a graphical presentation of Example D of a foamer evaluation according to an embodiment such as the system 200 illustrated in FIG. 5.

FIG. 13 is a graphical presentation of Example F showing cumulative hydrocarbon discharge over time at increasing gas rates of 5, 7, and 10 L/min according to an embodiment such as the system 300 illustrated in FIG. 6 or the system 400 illustrated in FIG. 7.

FIG. 14 is a graphical presentation of Example G showing cumulative hydrocarbon discharge over time at 5 and 7 L/min according to an embodiment such as the system 300 illustrated in FIG. 6 or the system 400 illustrated in FIG. 7.

FIG. 15 is an illustration of another embodiment of a system according to the disclosure, wherein a system 460 that is like the system 300 illustrated in FIG. 6 (with the same referenced elements) additionally includes a condenser 470 (for example, of the Graham type) where the condenser 470 has an outer jacket 472, a spiral coil conduit 474 for fluid (e.g., gas, foam, vapor, or condensate) to flow from an upper end of the condenser 470 down through the spiral coil conduit 474 as indicated by arrows 476 to a lower end of the condenser 470, and a water flow path through the outer jacket 472 and around the spiral coil conduit 474, where the condenser 470 additionally has a lower water port 482 at the lower end of the condenser 470 into the jacket 472 and an upper water port 484 at an upper end of the condenser 470, whereby cool water (for example, tap water) can be flowed into either the lower water port 482 or the upper water port 484 (for example, into the lower water port 482) and out the other water port (for example, out the upper water port 484) at the other end of the condenser 470 around the spiral coil conduit 474 for cooling the spiral coil conduit 474, where foam and condensate can be collected from the lower end of the spiral coil conduit 474 of the condenser 470 into the second graduated cylinder 342 on the mass balance 340. Preferably, the mass balance 340, the second graduated cylinder 342, and the condenser 470 are positioned in a laboratory fume hood 490, where the fume hood 490 is to reduce exposure of laboratory technicians to potentially toxic vapors or gases.

### DETAILED DESCRIPTION AND EXAMPLES

The disclosure will be described by referring to the general context for the systems and methods and to examples of how they can be made and used.

### Conventional System and Method of Foam Evaluation and Identified Disadvantages

A conventional system for evaluating a foamer consists of a graduated cylinder, one or more pipettes or syringes for measuring an amount of foamer, a mixer, and a stopwatch, where the measurement and evaluation in the conventional method is of foam half-life.

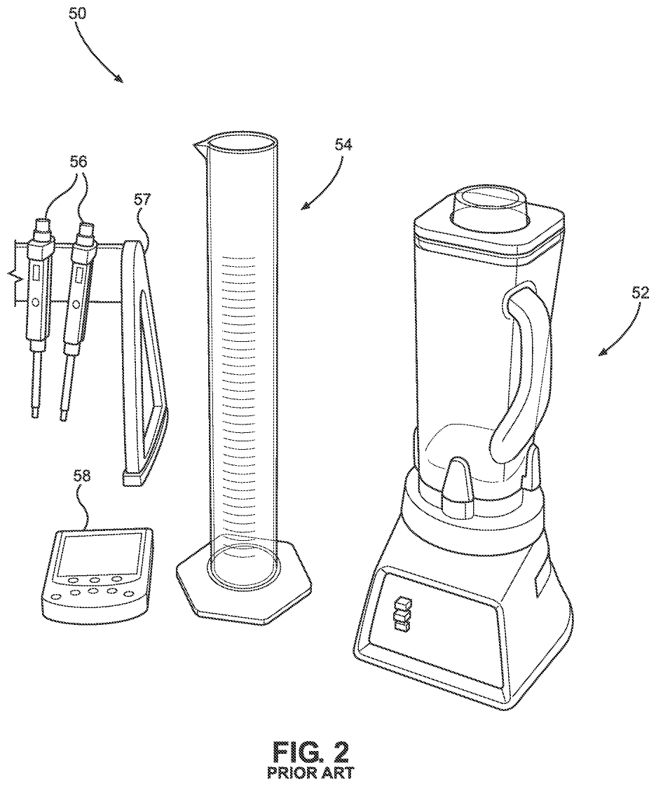

FIG. 2 is an illustration of a conventional (prior art) system 50 of laboratory equipment used for foam testing and evaluation, where the system 50 includes a blender or mixer 52, a graduated cylinder 54, one or more pipettes or syringes 56 (which can be optionally and conveniently held upright on a syringe stand 57), and a stopwatch 58.

According to the conventional system and method, the method includes steps of measuring out 300 mL of liquids at predetermined ratio of an aqueous liquid to a hydrocarbon liquid, pouring the measured amount of aqueous liquid into the mixer 52, adding a predetermined amount of a foamer to the aqueous liquid in the mixer 52 (using a pipette or syringe 56), mixing well, adding the hydrocarbon liquid, and mixing well again. The mixture of a foamer, an aqueous liquid, and a hydrocarbon liquid is subjected to shear in the mixer for 1 minute at 1,000 rpm. After mixing, a simple volumetric measurement is made in the graduated cylinder 54 of the total volume of the foam and liquid (that is, the measured volume at the top of a foam column in the graduated cylinder 54) and the time (using the stopwatch 58) for the total volume of the foam and liquid to be reduced by half.

As identified in this disclosure, advantages of the conventional system and method include (in no particular order): (a) it is a simple experiment to conduct; (b) the equipment is typically available in most field laboratories; (c) no compressed gas is required; and (d) it can give some relative performance evaluation.

As identified in this disclosure, disadvantages of the conventional method include (in no particular order): (a) shear in the mixing step can result in formation of stable emulsion between a liquid phase mixture of water and a hydrocarbon liquid; (b) it is not unusual for the results to be misinterpreted due to emulsion formation; (c) the foam half-life test is based purely on foam volumetric analysis, where the half-life is defined as the time taken for half of the foam volume created to be reduced (collapse); (d) there is no consideration given to foam density or foam mass; (e) the foam can maintain its volume while “dropping” a large fraction of liquid, thereby yielding a falsely optimistic foam evaluation; and (f) no temperature dependence is evaluated.

### Improved Systems and Methods

Improved systems and methods are provided to evaluate the performance of a foamer in unloading liquids such as water or condensate. The systems and methods reduce the potential to create stable emulsions caused by high mechanical shear of mixing in a mixer to create a foam. Additionally, the systems and methods described here avoid misleading interpretations from conventional systems and methods where only the foam half-life has been considered. Falsely optimistic results, from a foam half-life perspective, can occur where certain surfactants result in “weak” foams that can maintain volume while “dropping” a large fraction of the liquid out of the foam. In the conventional systems and methods, neither foam density nor foam mass versus time is considered.

The improved systems and methods described here provide an evaluation of a foamer based on measurements of the time to create a known volume of foam, the rates of foam dissipation and liquid phase recovery rates. This analysis is done by plotting several foam and liquid parameters versus time. In addition, measurements of the different phases can be made.

This disclosure will introduce new systems and methods to evaluate foamers for condensate or for produced water. This will help in optimizing the design and selection of foamers for use in various gas lift operations and hence help enhance hydrocarbon production.

Foamers can utilize different types of chemistry, such as hydrophilic/lipophilic (head/tail) or amphiphile (where the whole molecule has affinity for both oil and water). A hydrophilic portion of a foamer can be classified as anionic (−), cationic (+), amphoteric (zwitterionic) (+/−), or nonionic. Various types of foamers can be evaluated for use in a well, such as, amine oxides (cationic), betaines (amphoteric), quaternary amines (cationic), sulfated ether alcohols (anionic), sulfobetaines (amphoteric), and sulfonates (anionic).

The improved systems and methods are adapted for laboratory small-scale testing and evaluation, including the use of new combinations of laboratory equipment. The systems and methods are adapted for laboratory evaluation testing under simulated, steady conditions for a test of a foamer in a liquid simulating some of the downhole conditions observed or expected in a well. In various embodiments, each laboratory test is a discrete and separate test under discrete and separate test conditions from another test. For example, the composition of the liquid being sparged can be different or the sparging conditions can be different. The sparging conditions can include, for example, the type of gas, the gas flow rate, the characteristics of the gas delivery tubing, the characteristics of the frit for diffusing gas, and the temperature of a temperature-controlled bath for the sparging.

According to various embodiments of the present disclosure, an objective is to have a measure for the ability of a foamer to affect the removal of water or condensate (also known as unloading) from the tubulars in a gas-producing well. The interest is in determining how much liquid can be removed from the tubulars in a well, not about the foam. In other words, the interest is in the liquid, not in the foam. But the conventional system and method does not measure the liquid, the conventional system and method measures a volume of the foam and the half-life of the foam. This does not consider the volume of liquid dropping out of the foam or that the foam could be of high density or of low density depending on the liquid content.

### Basic Improved Systems and Methods

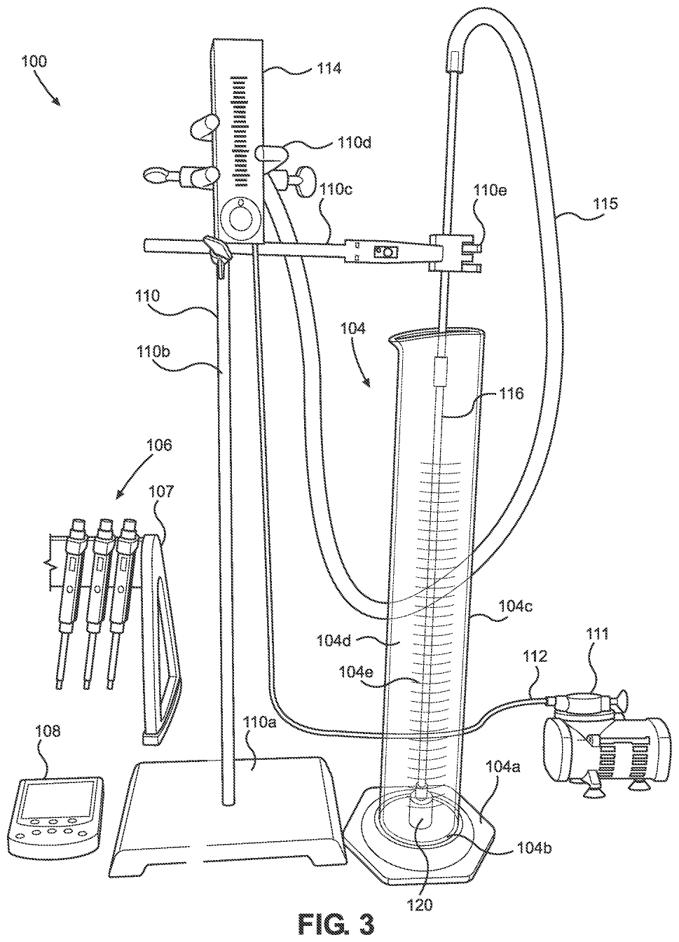

FIG. 3 is an illustration of a basic system 100 according to an embodiment of the present disclosure, where the system 100 includes graduated cylinder 104, one or more pipettes or syringes 106 (which can optionally and conveniently be held upright on a syringe stand 107), a stopwatch 108, a laboratory stand 110, a gas source, such as an air, a variable rate gas compressor 111, a first gas tubing 112 operatively connecting from the gas compressor 111 to a gas flow meter 114 conveniently supported by the laboratory stand 110, a second gas tubing 115 operatively connecting from the gas flow meter 114 to gas delivery tubing 116 having a frit 120 for sparging a gas down into the graduated cylinder 104.

The graduated cylinder 104 has a base 104a, a flat interior bottom 104b, a cylindrical wall 104c, an interior cylindrical volume 104d, and graduated markings 104e on the exterior of the cylindrical wall 104c. The material of the graduated cylinder 104 should be transparent for seeing into the graduated cylinder 104. The graduated cylinder 104 can be made of glass or plastic, although glass is preferred. The graduated cylinder 104 is of an appropriate size, preferably 1,000 mL capacity.

The laboratory stand 110 has a base 110a, an upright support 110b, an arm 110c, a clamp 110d for holding the gas flow meter 114, and second clamp 110e. The height of the arm 110c is adjustable on the stand 110.

The gas flow meter 114 is conveniently supported by the clamp 110d on the upright support 110b of the stand 110. The gas flow meter 114 can be of any suitable type, such as a bubble meter having markings for reading a flow rate of a gas through the bubble meter. The gas delivery tubing 116 is conveniently supported by the clamp 110e on the arm 110c of the stand 100.

In various embodiments, the first gas tubing 112 and the second gas tubing 115 is preferably flexible.

The gas delivery tubing 116 is rigid and is operatively connected to or has the frit 120 of porous material at the end of the gas delivery tubing 116. The gas delivery tubing 116 can be of an appropriate length and an appropriate internal diameter for conducting the desired rate of gas flow. The frit 120 can be of a desired and appropriate material, shape, size, and porosity for conducting the desired rate of gas flow through the frit 120 and forming a desired size of initial bubbles in the liquid in the graduated cylinder 104. In various embodiments, these characteristics of the gas delivery tubing 116 and the frit 120 are selected to be the same in making comparative tests of different foamers, concentrations, liquid compositions, or conditions of sparging for foaming. In various embodiments, the position or depth of the frit 120 in the liquid can be adjusted as may be desired. In various embodiments, the frit 120 is positioned at or adjacent to the bottom of the graduated cylinder 104. In various embodiments, the gas delivery tubing 116 and the frit 120 can be easily removed from the graduated cylinder 104 for cleaning, such that no residue of a previous test of a foamer and a liquid should remain that would interfere with a test and alter the results of a subsequent test.

The graduated cylinder 104 is for measuring volumes of fluids, such as a volume of a liquid in which a foamer is to be evaluated and the volume of a foam created by sparging the liquid. The liquid can be made of water, brine, hydrocarbon, or a combination thereof.

The pipettes or syringes 106 are used for measuring out a volume of a foamer to be evaluated in a test of a liquid using the system 100. Micropipettes with disposable tips or disposable syringes 106 are preferable.

The stopwatch 108 can be of any convenient type.

A gas source such as the gas compressor 111 is used to compress ambient air for use in a test using the system 100 according to various methods of this disclosure. In various embodiments, compressed air, nitrogen, or carbon dioxide from a gas tank can be used instead of a gas compressor 111 using ambient air. Compressed air or nitrogen or carbon dioxide is used to simulate a gas stream in a well. The compressed gas, whether air, nitrogen, or carbon dioxide, is controlled to flow through the first gas tubing 112, through the gas flow meter 114 for measuring the flow rate, through the second gas tubing 115 to flow through the gas delivery tubing 116, and out through a diffuser or sparge (e.g., the frit 120) at or near the bottom 104b of the graduated cylinder 104, which then can flow up through the liquid containing foamer in the graduated cylinder 104 at a predetermined gas flow rate. This sparging of gas through the liquid can create a foam of the liquid and the gas in a column laying above any remaining volume of the liquid mixture at the bottom of the graduated cylinder 104.

For example, the gas delivery tubing 116 for sparging the liquid can be a glass or stainless steel sparge tube having a suitable glass or aluminum frit 120 affixed at the end of the tube 116.

In various embodiments, a gas flow meter 114 having an appropriate flow measurement capacity can be used, for example, flow meters can have ranges up to 2, 5, or 28 L/min for various testing parameters. For example, foaming of hydrocarbons can require higher gas flow rates than foaming of aqueous liquids.

In a basic method using the system 100 according to this disclosure, a total volume (e.g., 200 mL) of liquid or liquids at predetermined ratio of aqueous phase to hydrocarbon phase is measured out for use in a test of a foamer for the liquid or a mixture of liquids. The liquid can be either an aqueous liquid (e.g., water or a brine), a hydrocarbon liquid (e.g., an oil, such as a condensate), or a predetermined combination of these. In addition, a foamer for evaluation can be added in a predetermined concentration. In various embodiments, depending on the foamer a predetermined amount of the foamer is added to the aqueous phase, mixed well, then the hydrocarbon liquid is added and mixed. The liquids for a mixture can be mixed directly in the graduated cylinder 104 or later poured into the graduated cylinder 104 after mixing. Mixing in a blender or mixer is not required. Then insert the gas delivery tubing 116 with the frit 120 at the end is positioned in the graduated cylinder 104 such that the frit 120 is positioned at or adjacent to the bottom 104b of the graduated cylinder 104 and preferably such that the frit 120 is completely submerged in the liquid at the bottom of the graduated cylinder. Then the gas compressor 211 is started (or the gas flow from a pressurized gas tank is started) to flow the gas at a predetermined rate through the liquid mixture and the stopwatch 108 is started at the same time. The elapsed time is measured with the stopwatch 108 until foaming reaches up to the 1,000 mL volume mark of the graduated markings 104e of the graduated cylinder 104. The time for foaming to fill the graduated cylinder 104 is referred to as the “purge time”). At the purge time, the flow of gas is stopped, for example, by switching off the gas compressor 111. After the purge time, the volume of liquid recovered from the foam and the remaining foam volume, as a function of time, are measured and recorded.

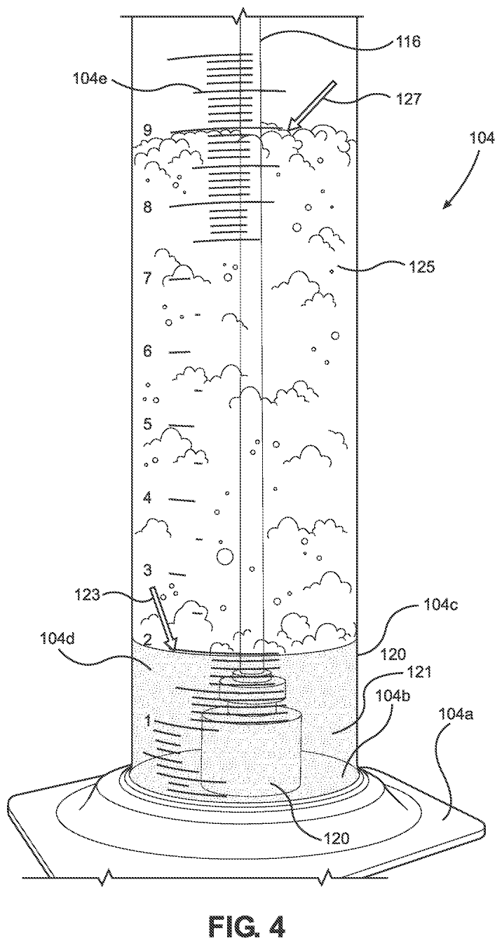

FIG. 4 is an illustration of an example observation and measurement that can be made in the graduated cylinder 104 of the system 100 of FIG. 3, where after initially foaming of a liquid by sparging with a gas (from the gas compressor 111, not shown in FIG. 4) via the gas delivery tubing 116 down to the frit 120 positioned at or adjacent to the bottom 104b of the graduated cylinder 104, the volume of a liquid column 121 at the bottom of the graduated cylinder 104 can be measured in the graduated cylinder at arrow 123 using the graduated markings 104e, and further where the total volume of the liquid column 121 plus a foam column 125 can be measured in the graduated cylinder 104 at arrow 127 using the graduated markings 104e. The graduated markings 104e can include, for example, volume measurement markings of 1, 2, 3, 4, 5, 6, 7, 8, 9 that are graduated from the bottom 104b of the graduated cylinder 104 upwards in increments of 100 mL (and unlabeled subdivisions in increments of 10 mL) in the case of a graduated cylinder 104 having a total volume of 1,000 mL.

It should be understood, of course, that a liquid column 121 would be at the bottom of the graduated cylinder 104 and any foam column 125 would be above the liquid column 121 because a liquid would have a higher density than a foam formed of the liquid and gas. In addition, the volume of the liquid column 121 and the foam column 125 can be separately measured, where the volume of the foam column 127 would be the total volume less the volume of the liquid column 121. Further, visual observations of the characteristics of the liquid in the liquid column 121 and the foam of the foam column 125 can be made. Such observations and measurements can be made periodically over time and recorded or graphed (not shown in FIG. 4).

According to various embodiments of the improved systems and methods, the liquid phase of the liquid column 121 at the bottom of the graduated cylinder 104 is measured, the rate at which liquid is recovered from the foam after sparging is measured, and how long it takes for half of that liquid to be returned into the graduated cylinder is measured. This is a measurement of interest. This can provide a better indication of the foam stability and a realistic way to establish relative foamer stability performance when comparing different foamers or foamers at different concentrations or the performance of a particular foamer at different temperatures.

Like in the conventional system and method, it is possible to make a simple volumetric foam analysis of the foam half-life, that is, to measure the time taken for the foam volume created to be reduced by half. Advantages of this basic system and method include (in no particular order): (a) it is a relatively simple experiment to conduct; (b) the equipment is typically available in most field laboratories; (c) the creation of a stable emulsion is less likely by not using high shear in a mixer; and (d) the foam half-life can give some relative performance evaluation, like in the conventional system and method described above. Disadvantages of this basic system and method include (in no particular order): (a) a compressed gas is required; (b) the foam half-life test is based purely on foam volumetric analysis; (c) there is no consideration given to foam density or foam mass; (d) the foam can maintain its volume while “dropping” a large fraction of the liquid fraction, thereby yielding a falsely optimistic result; and (d) no temperature dependence is evaluated here. The simple volumetric analysis of foam half-life is an indicator of performance, but the performance is still dependent upon other factors not taken into account or measured.

However, in addition to a simple volumetric measurement of foam half-life after using compressed gas flow through the liquid, foamer evaluation according to the improved systems and methods disclosed herein can include one or more of the following measurements: (a) time to create a known volume of foam with the gas flow through the liquid mixture, (b) rate of liquid recovery (“liquid half-life”), (c) foam density profile over time, (d) visual observation of the separate phase attributes, and (e) relative performance established through simultaneous consideration of more than one of these measurements.

When comparing to a real well scenario, it is upward flow velocity (gas flow rate/cross sectional area) and not rate alone that is an important parameter. The tests according to the basic systems and methods of this disclosure are conducted at atmospheric pressure. In this regard, gas compression under downhole conditions should be considered when calculating downhole gas velocities if a comparison to actual well flowing conditions is desired. This is complicated by the fact that in the laboratory a continuous gas flow is used with a fixed liquid volume. Nevertheless, the testing according to the disclosed systems and methods is more realistic than the conventional system and method.

The following are some of the factors that can influence the outcome of a particular test (in no particular order): (a) fluid sources, (b) salinity of an aqueous solution (e.g., a brine), (c) phase volume fractions, (d) gas flow rate, (e) foamer concentration, (g) temperature, and (h) sparge tube selection.

The following are some of the design considerations for a test according to the methods (in no particular order): (a) gas or oil well, (b) gas production rate, (c) expected gas lift rate, (d) if an oil well, the water cut, (e) the salinity of the produced water, (f) samples of fluids from the well site, (g) tubular sizes in the well, (h) downhole temperature, (i) desired key performance indicators (“KPIs”) for product performance.

Improved Systems and Methods with Temperature Control

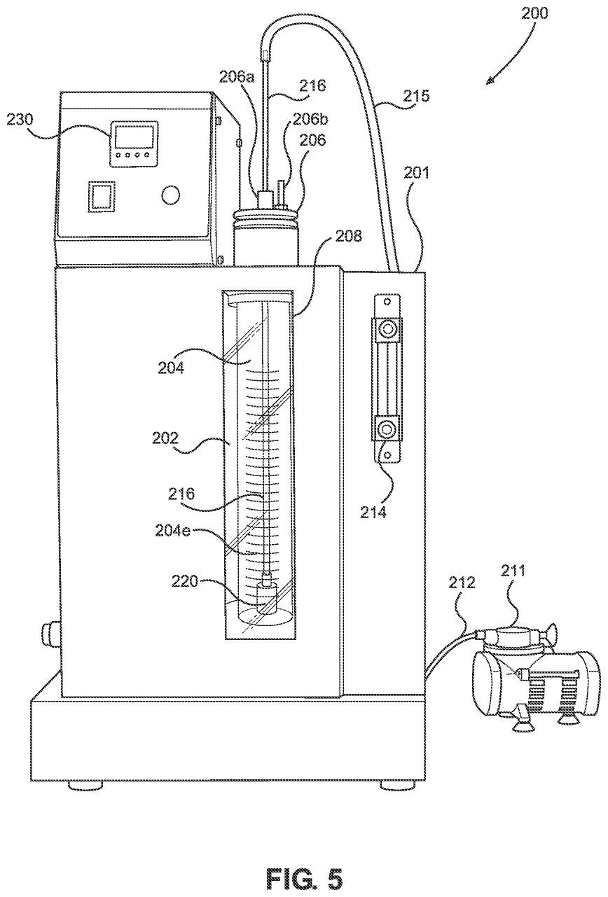

FIG. 5 is an illustration of an embodiment of a system 200 according to the disclosure that includes a temperature-controlled bath apparatus 201, where the temperature-controlled bath apparatus 201 has a temperature-controlled bath 202 of a liquid medium (such as water) that can be heated to a predetermined temperature, a graduated cylinder 204 (preferably like the graduated cylinder 104 illustrated in FIG. 4), where the graduated cylinder 204 can be positioned down into and substantially submerged in the liquid medium of the bath 202, a transparent window 208 (preferably of glass) to see into the temperature-controlled bath 202 and to the graduated cylinder 204, where the graduated cylinder 204 has graduated markings 204e and where the temperature-controlled bath apparatus 201 preferably has a backlight (not shown in FIG. 5) so that the graduated cylinder 204 can be backlit in the temperature-controlled bath 202, a gas flow meter 214, which can be optionally and conveniently built in the temperature-controlled apparatus 201, and a control panel 230 used for controlling the temperature of the temperature-controlled bath 202. In this system 200, the graduated cylinder 204 preferably additionally has a sealable lid 206, a first port 206a through the sealable lid 206, and a second port 206b through the sealable lid 206. In this system 200, a gas source, such as an air compressor 211, is operatively connected by a first gas tubing 212 (going behind and into the apparatus 201) and to the built-in gas flow meter 214 of the temperature-controlled bath apparatus 201, and the gas flow meter 214 is operatively connected by a second gas tubing 215 to a gas delivery tubing 216 that can be positioned to go down through the first port 206a in the lid 206 for the graduated cylinder 204 to position a frit 220 at or near the bottom of the graduated cylinder 204.

The temperature-controlled bath 202 of the apparatus 201 preferably uses a liquid for the bath that is substantially transparent so that it is possible to see through the transparent window 208, through the substantially transparent liquid of the temperature-controlled bath 202, through the transparent material of the graduated cylinder 204, and into the graduated cylinder 204. Examples of liquids that are substantially transparent include water and mineral oil. This allows for the measuring of the liquid volume (liquid column), the foam volume (foam column), or both in the graduated cylinder 204.

The first port 206a in the lid 206 can be used to position and support the gas delivery tubing 216 in the graduated cylinder 204. In this embodiment, the second port 206b through the sealable lid 206 can be left open to the atmosphere so gas can leave the graduated cylinder 204. The gas flow meter 214 can be of any suitable type, for example, a bubble meter. The first gas tubing 212 and the second gas tubing 215 are preferably flexible. The gas delivery tubing 216 can be rigid and made of a material such as glass or stainless steel. Observations and measurements like those illustrated in FIG. 4 can be made by viewing through the transparent window 208, through the liquid medium of the temperature-controlled bath 202, and into the graduated cylinder 204.

The graduated cylinder 204 in the system 200 can be similar or the same as the graduated cylinder 104 illustrated in the system 100 in FIG. 3.

Improved Systems and Methods with Collecting Foam in a Second Vessel

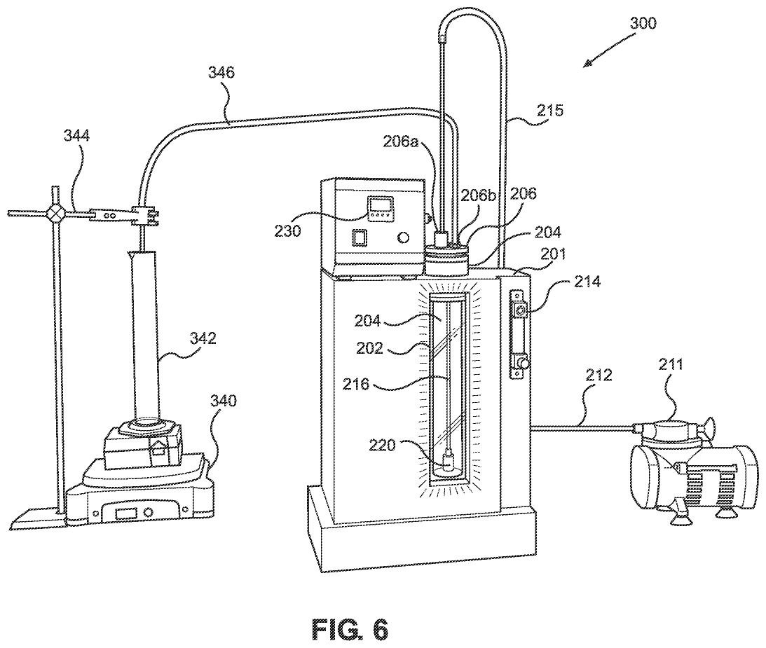

FIG. 6 is an illustration of another embodiment of a system according to the disclosure, wherein a system 300 that is like the system 200 illustrated in FIG. 5 (with the same referenced elements) additionally includes a mass balance 340, a second graduated cylinder 342 positioned on the mass balance 340, a laboratory stand 344 to support a third gas tubing 346, where the third gas tubing 346 is operatively connected to the second port 206b of the lid 206 for the graduated cylinder 204. The third gas tubing 346 is for conducting gas or foam from the graduated cylinder 204 and delivering it such that any foam or any liquid dropped from the foam can have the volume measured by the second graduated cylinder 342 or the mass measured by the mass balance 340.

In the system 300, the second graduated cylinder 342 can be like the first graduated cylinder 204 (and like the graduated cylinder 104 as illustrated in FIG. 3 and FIG. 4). In the alternative to a second graduated cylinder 342, another type of appropriate laboratory vessel can be used, for example, a graduated beaker or a flask. The laboratory stand 344 can be similar to the laboratory stand illustrated in FIG. 3, having a base, an upright support, an arm, and one or more appropriate connecting clamps. The third gas tubing 346 is preferably flexible.

In various preferred embodiments of the system 300, the mass balance 340 can be positioned in a laboratory fume hood (not shown) for safety of a laboratory technician operating the system 300. The mass balance is connected to a computer to allow continuous data logging (mass of liquid evacuated from the foam column) vs. time.

Improved Systems and Methods with Camera Recording and Computer Analysis

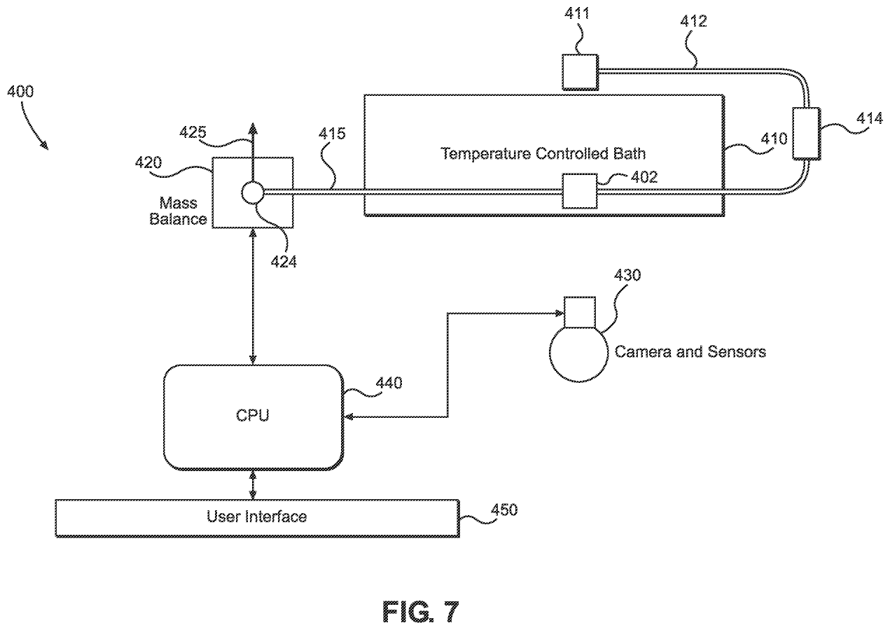

FIG. 7 is a schematic of a system 400 like the system 300 illustrated in FIG. 6 but additionally showing a camera and computer control, where the system 400 includes a temperature-controlled bath 410 for a first vessel 402, a gas source 411, a gas tubing 412 operatively connected from the gas source 411 through a gas flow meter 414 and from there the gas tubing 412 operatively connected to continue from the gas flow meter 414 to and down into the first vessel 402 for sparging a liquid in the first vessel 402 with a gas from the gas source 411, a fluid tubing 415 from the first vessel 402 in the temperature-controlled bath 410 to a second vessel 424 positioned on a mass balance 420, at least one camera 430 for taking pictures or a video recording of the appearance of a liquid or foam in the first vessel 402 and for measuring the volume of a liquid or of a foam vs. time or of the total volume of liquid and foam in the first vessel 402 of the temperature-controlled bath 410 (and optionally one or more other sensors with the camera 430), a central processing unit (“CPU”) 440, where the CPU 440 is operatively connected to obtain and record mass measurements from the mass balance 420, where the CPU 440 is additionally operatively connected to control or receive images or information data from the camera 430 and any optional sensors, and where the CPU 440 has a user interface 450 operatively connected to the CPU 440. The user interface 450 can be, for example, a computer screen or a printer. As any foam is collected from the first vessel 402 into the second vessel 424 and as the foam column collapses in the second vessel 424, the entrained liquid in the foam collects in the second vessel 424 and the gas from the foam can flow out of an upper opening 425 of the second vessel 424.

In various embodiments of the system 400, the gas source 411 can be an air compressor for compressing ambient air. In various embodiments, the gas source 411 can be a tank of a compressed gas, such as a tank of compressed air, nitrogen, or carbon dioxide.

The gas tubing 412 is adapted to deliver the gas from the gas source 411 to at or near the bottom of the first vessel 402, which can be like as shown in FIG. 6 for the gas flow from the gas compressor 211, through the first gas tubing 212, through a gas flow meter 214, through the second gas tubing 215 to the gas delivery tubing 116 from the gas flow meter 114 to and down into the graduated cylinder 204, and to the frit 220 of porous material at the end of the gas delivery tubing 216 and positioned at or adjacent to the bottom of the graduated cylinder 204.

Referring again to FIG. 7, the second vessel 424 can be any suitable vessel, such as a graduated cylinder or beaker for containing a fluid or liquid and having an open top to allow for gas to escape leaving the liquid from the foam in the second vessel 424 for weighing on the mass balance 420.

Optionally, the CPU 440 can be additionally operatively connected by appropriate control lines to one or more of the temperature-controlled baths 410, the gas source 411, the gas tubing 412, and the fluid tubing 415. The purpose of such connections would be to provide computer control for starting and stopping the gas flow and for controlling the gas flow rate by the CPU 440.

In various embodiments, the temperature-controlled bath 410 and the first vessel 402 can be like the temperature-controlled bath apparatus 201 with a graduated cylinder 204 as in FIG. 5.

In various preferred embodiments according to the system 400 illustrated in FIG. 7, the following types of data can be measured by a mass balance 420 or by the camera 430 and automatically logged versus time (in no particular order): (a) foam level (volume); (b) liquid level (volume); (c) total mass of liquid unloaded (e.g., water in case of testing and evaluating a foamer for water unloading or a hydrocarbon liquid in case of testing and evaluating a foamer for condensate). In various preferred embodiments, a test can be video recorded and logged from the video.

A purpose of the camera or cameras is to data log volume of foam and volume of liquid versus time. That data is logged by playing back the video frame by frame after an experiment has been completed and reading off the volumes from the screen. Without a camera, it is virtually impossible to capture this data live or “real time” because the changes in volume are happening too quickly to manually log real time.

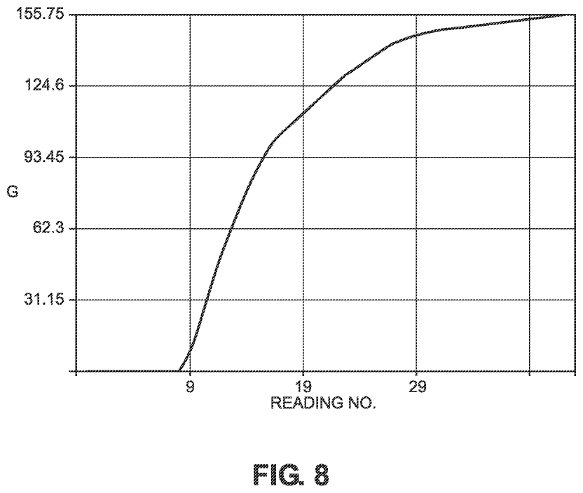

FIG. 8 is an example of a graph of test data that can be manually recorded with the system 300 illustrated in FIG. 6 or automatically recorded with the system 400 illustrated in FIG. 7, where the graph of FIG. 8 shows the amount of foam or liquid (in grams) collected in the second graduated cylinder 342 on the mass balance 340 of the system 300 illustrated in FIG. 6 or collected in the second vessel 424 on the mass balance 420 of the system 400 illustrated in FIG. 7 (in measurement readings over time, e.g., every 2 to 5 seconds). The mass of the foam is essentially that of the liquid of the foam because the mass of the gas of the foam is negligible.

Further, foam density measurements can be made. For example, as illustrated in FIG. 4, the foam column 125 is visible toward the top of the graduated cylinder 104. Similarly, such measurements can be made with the system 400. Starting with 200 mL of liquid and shutting of the gas flow at 16 seconds obtained 1,000 mL of foam and 25 mL of liquid left. Knowing the density of the liquid allows for calculation of the average foam density. For example, if the liquid were essentially pure water, the density (or specific gravity) of the liquid is about 1.0 g/mL. Having 175 mL of water in 975 mL of foam volume gives a calculated foam density of 179.5 g/L. For another example, if the liquid is a brine having a specific gravity of 1.0955 g/mL, having 175 mL of brine in 975 mL of foam volume gives a calculated foam density of 196.6 g/mL. The next step is to observe and make that the density measurement continuously over time as the liquid level recovers at the bottom of the graduated cylinder.

### Example A—Evaluation of Foamer X in Water

An example of an evaluation test of a water-based (hydrophilic) Foamer X is provided as Example A. In this Example A, the foamer is tested with a synthetic brine without any hydrocarbon liquid. In this example, water with Foamer X at a concentration of 5 gallon per thousand is used with gas sparging to create a foam in a graduated cylinder, in a system 100 as illustrated in FIG. 3. Table A1 summarizes the test conditions used in this Example A.

TABLE A1
Example A Test Conditions

| Total Fluid Volume | 200 | mL |
|---|---|---|
| Aqueous phase | Synthetic | |
| Fraction | 100% | |
| Volume | 200 | mL |
| TDS (Total Dissolved Solids) | 10,000 | ppm |
| SG (Specific Gravity) | 1.0955 | |
| Hydrocarbon phase | None | |
| Fraction | 0% | |
| Volume | 0 | mL |
| SG (Specific Gravity) | N/A | |
| API | N/A | |
| Foamer | A | |
| Concentration | 5 | gpt |
| Concentration fraction of volume | Total | |
| Volume | 1 | mL |
| Temperature (about room temperature) | 23.3° C. (73.94° F.) | |
| Gas | Compressed Air | |
| Gas flow rate(s) | 900 | mL/min |

| | |
|---|---|
| Sparge Material | Glass |

An example of a foam density profile over time cab be obtained as shown in Table A2, where the volume measurements are made like for the illustration of FIG. 4 and over time as measured in the system 100 with a stopwatch 108 as illustrated in FIG. 3. In this Example A, the test is at an ambient room temperature of 23.3° C. (73.94° F.) so a system having controlled-temperature bath is not required. For any particular test, in that context the “liquid” is any aqueous phase, any hydrocarbon phase, and any foamer that is mixed together and forms the liquid column 121 as illustrated in FIG. 4. Similarly, in the context of a particular test, the “foam” is any foam column 125 as illustrated in FIG. 4 as formed by sparging with a gas using the system 100 of FIG. 3. The measurements can include, with reference to FIG. 4 for the methodology of volume measurements:
(a) “Event;
(b) “Time (minutes:seconds)”;
(c) “Delta Time (seconds), that is, the time measured from the time of the start event of gas on;
(d) “Liquid Volume (mL)” (also referred to herein as “Liquid Hold Up (mL)”) like measured at the arrow 123 as the liquid level at the top of the liquid column 121;
(e) “Total Volume of Liquid and Foam (mL)” like measured at the arrow 127 at the top of the foam column 125;
(f) “Foam Volume (mL),” that is, the volume of only the foam column 125 between the arrows 123 and 125;
(g) “Calculated Liquid % not in Foam” (also referred to herein as “Hold Up Retained %”) that is, the percent of the original liquid volume at the time of the gas on that is remaining in the liquid volume;
(h) “Calculated Liquid (mL) in Foam,” that is, the volume of liquid foamed from the liquid volume from the original liquid volume at the event time of gas on; and
(i) “Calculated Average Foam Density (g/L),” that is, the “Calculated Liquid (mL) in Foam” X a known specific gravity (“SG”) for the liquid/“Foam Volume (mL)”, where this is an average density over the entire Foam Volume (mL), regardless of any foam density variations within the foam column 125.

TABLE A2
Example A Test Data

| | | | Liquid Vol. | Total | | Calc. Liq. | Calc. | Calc. |
|---|---|---|---|---|---|---|---|---|
| | | | (mL) (aka | Vol. of | | (%) not in | Liq. | Avg. |
| | | Delta | “Liq. | Liquid | Foam | Foam (aka | (mL) | Foam |
| | Time | Time | Hold Up | and Foam | Vol. | “Hold Up | in | Dens |
| Event | (min:sec) | (sec) | (mL)”) | (mL) | (mL) | Retained %”) | Foam | (g/L) |
| Gas On | 00:21.7 | 0.00 | 200 | 200 | 0 | 100 | 0 | NA |
| | 00:40.0 | 18.30 | 190 | 270 | 80 | 95 | 10 | 136.9 |
| | 00:46.5 | 24.80 | 180 | 300 | 120 | 90 | 20 | 182.6 |
| | 00:53.3 | 31.60 | 170 | 350 | 180 | 85 | 30 | 182.6 |
| | 00:59.5 | 37.80 | 160 | 400 | 240 | 80 | 40 | 182.6 |
| | 01:05.9 | 44.20 | 150 | 450 | 300 | 75 | 50 | 182.6 |
| | 01:12.2 | 50.50 | 140 | 490 | 350 | 70 | 60 | 187.8 |
| | 01:18.5 | 56.80 | 130 | 530 | 400 | 65 | 70 | 191.7 |
| | 01:23.8 | 62.10 | 120 | 570 | 450 | 60 | 80 | 194.8 |
| | 01:29.6 | 67.90 | 110 | 610 | 500 | 55 | 90 | 197.2 |
| | 01:34.2 | 72.50 | 100 | 645 | 545 | 50 | 100 | 201.0 |
| | 01:38.7 | 77.00 | 90 | 685 | 595 | 45 | 110 | 202.5 |
| | 01:43.2 | 81.50 | 80 | 715 | 635 | 40 | 120 | 207.0 |
| | 01:47.6 | 85.90 | 70 | 735 | 665 | 35 | 130 | 214.2 |
| | 01:52.6 | 90.90 | 60 | 785 | 725 | 30 | 140 | 211.5 |
| | 01:57.9 | 96.20 | 50 | 825 | 775 | 25 | 150 | 212.0 |
| | 02:05.9 | 104.20 | 40 | 885 | 845 | 20 | 160 | 207.4 |
| | 02:15.1 | 113.40 | 30 | 960 | 930 | 15 | 170 | 200.3 |
| Gas Off | 02:18.6 | 116.90 | 25 | 1000 | 975 | 12.5 | 175 | 196.6 |
| | 02:24.9 | 123.20 | 30 | 1000 | 970 | 15 | 170 | 192.0 |
| | 02:36.5 | 134.80 | 40 | 1000 | 960 | 20 | 160 | 182.6 |
| | 02:49.4 | 147.70 | 50 | 1000 | 950 | 25 | 150 | 173.0 |
| | 03:01.5 | 159.80 | 60 | 1000 | 940 | 30 | 140 | 163.2 |
| | 03:12.8 | 171.10 | 70 | 1000 | 930 | 35 | 130 | 153.1 |
| | 03:27.1 | 185.40 | 80 | 1000 | 920 | 40 | 120 | 142.9 |
| | 03:43.6 | 201.90 | 90 | 1000 | 910 | 45 | 110 | 132.4 |
| | 03:59.9 | 218.20 | 100 | 1000 | 900 | 50 | 100 | 121.7 |
| | 04:18.9 | 237.20 | 110 | 1000 | 890 | 55 | 90 | 110.8 |
| | 04:37.5 | 255.80 | 120 | 1000 | 880 | 60 | 80 | 99.6 |
| | 05:04.8 | 283.10 | 130 | 1000 | 870 | 65 | 70 | 88.1 |
| | 05:30.0 | 308.30 | 140 | 1000 | 860 | 70 | 60 | 76.4 |
| | 05:59.5 | 337.80 | 150 | 1000 | 850 | 75 | 50 | 64.4 |
| | 06:31.5 | 369.80 | 160 | 1000 | 840 | 80 | 40 | 52.2 |
| | 07:17.7 | 416.00 | 170 | 1000 | 830 | 85 | 30 | 39.6 |
| | 08:15.6 | 473.90 | 180 | 1000 | 820 | 90 | 20 | 26.7 |
| End Test | 10:10.5 | 588.80 | 190 | 980 | 790 | 95 | 10 | 13.9 |

As can be seen from the data in Table A2 for the Example A, the foam density changes over time. After stopping the gas flow, the liquid is dropping out from the foam and the decreasing foam density change is a better measure of foam stability than foam volume. This data provides the rate at which the foam density changes. In addition, visual observations of the liquid column and the foam column can be made. This type of testing and evaluation as illustrated by this Example A can be done for various concentrations of a foamer in various liquid systems of interest, such as aqueous based, hydrocarbon-based, or combinations, with or without a foamer, and where the proportions can be varied and the concentration of foamer can be varied.

Based on the data in Table A2 for Example A, the calculated “t(½) Foam Volume (mL)” would be a boundary calculated as one-half of the Foam Volume (mL) at the purge time of stopping the gas flow. In this example, this is calculated to be 487.5 mL, that is, one-half of the Foam Volume of 975 mL at the purge time (minutes:seconds) of 02:18.6. This “t(½) Foam Volume (mL)” can be used as a boundary for interpretation of the data and evaluating the performance of the foamer in the liquid under the test conditions. This calculated boundary “t(½) Foam Volume (mL)” is at what volume the foam created during the purge time would be one-half collapsed in volume.

In addition, based on the data in Table A2 for Example A, the “t(½) Liquid Volume (%)” (for the liquid volume not in the foam) would be a boundary calculated based on the Liquid Volume (%). In this example, this is calculated to be 56.25%, that is, “Liquid Volume % not in Foam” of 12.5% at purge time+(100% at start of gas on−12.5% at purge time)/2. This “t(½) Foam Volume (%)” (for the liquid volume % not in the foam) can be used as a boundary for interpretation of the data and evaluating the performance of the foamer in the liquid under the test conditions. This calculated boundary “t(½) Liquid Volume (%)” (for the liquid volume not in the foam) can be another boundary for evaluation of foam stability.

Further, based on the data in Table A2 for Example A, the “t(½) Foam Density (g/L)” would be a boundary calculated based on the Calculated Average Foam Density (g/L). In this example, this is calculated to be 98.3 g/L, that is, the one-half of the maximum “Calculated Average Foam Density (g/L)” of 196.5 g/L obtained in the test. This calculated boundary “t(½) Foam Density (g/L)” can be another bounder for evaluation of the performance of a foamer.

Table A3 summarizes the data and evaluation boundaries and the occurrences that can be determined from the measurements presented in Table A2.

TABLE A3
Example A Evaluation Boundaries

| | | Liquid Vol. | Total | | Liq. | |
|---|---|---|---|---|---|---|
| | | (mL) (aka | Vol. of | | Vol. | Avg. |
| | Delta | Liq. | Liquid | Foam | (%) | Foam |
| | Time | Hold Up | and Foam | Vol. | not in | Density |
| Event | (sec) | (mL)”) | (mL) | (mL) | foam | (g/L) |
| Gas On | 0 | 200 | 200 | 0 | 100 | N/A |
| Gas Off | 116.9 | 25 | 1000 | 975 | 12.5 | 196.6 |
| End | 588.8 | 190 | 980 | 790 | 95 | 13.9 |
| t(½) Liquid Volume (%) | 242 | 112.5 | N/A | N/A | 56.25 | N/A |
| t(½) Foam Volume (mL) | N/A | N/A | N/A | 487.5 | N/A | N/A |
| t(½) Avg. Foam Density (g/L) | 259 | N/A | N/A | N/A | N/A | 98.3 |

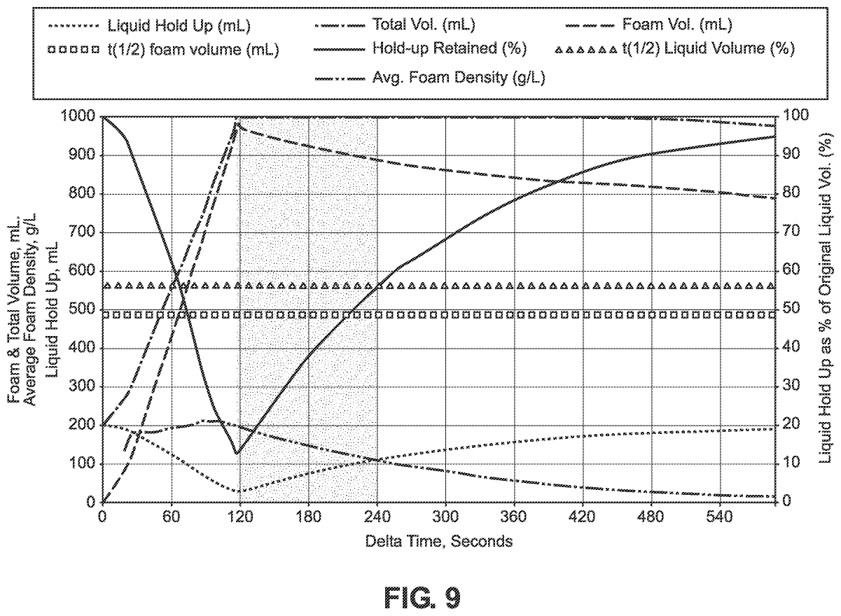

FIG. 9 is a graphical presentation of the data from Table A2 and Table A3 for Example A. The gas flow is on starting at Delta time of 0 seconds to 116.9 seconds. The gas off is at 116.9 seconds. The data from Table A2 is graphically presented in FIG. 9. The calculated boundary for the “t(½) Foam Volume (mL)” is shown in the graph of FIG. 9 to help with interpretation of the data results. In addition, the calculated boundary for the “t(½) Liquid Volume (%)” is shown in the graph of FIG. 9 to help with interpretation of the data results for Example A. The calculated boundary for the “t(½) Foam Density (g/L)” is not shown.

As can be determined graphically from FIG. 9, the “t(½) Liquid Volume (%)” of 56.25% for Example A occurs at 242 seconds, which is 125 seconds after gas off time of 116.9 seconds. Therefore, in this Example A the t(½) Liquid Volume (%) occurs at 125 seconds after gas off.

As can be determined graphically from FIG. 9, the “t(½) Foam Volume (mL)” of 487.5 mL for Example A does not ever occur at all during the test. By conventional measurement methods and standards, this would have indicated a good foamer result.

As can be determined graphically from FIG. 9, the t(½) Foam Density (g/L) of 98.3 g/L for Example A occurs at 259 seconds, which is 142 seconds after gas off time of 116.9 seconds. Therefore, the t(½) Foam Density (g/L) occurs at 142 seconds after gas off.

### Example B—Foamer Y in Water

An example of a test of another water-based (hydrophilic) Foamer Y is provided in Example B. In this Example B, the Foamer Y is tested with a sample of a produced water from a well without any added hydrocarbon liquid. In this example, water with Foamer Y at a concentration of 10 gallon per thousand is used with gas sparging to create a foam in a graduated cylinder, in a system 200 with a temperature-controlled bath apparatus 201 as illustrated in FIG. 5. Table B1 summarizes the test conditions used in this Example B.

TABLE B1
Example B Test Conditions

| Total Fluid Volume | 200 | mL |
|---|---|---|
| Aqueous phase | Sample of a Produced Water | |
| Fraction | 100% | |
| Volume | 200 | mL |
| TDS (Total Dissolved Solids) | N/A | |
| SG (Specific Gravity) | 1.163 | |
| Hydrocarbon phase | None | |
| Fraction | 0% | |
| Volume | 0 | mL |
| SG (Specific Gravity) | N/A | |
| API | N/A | |
| Foamer | Foamer Y | |
| Concentration | 10 | gpt |
| Concentration fraction of volume | Total | |
| Volume | 2 | mL |
| Temperature (Controlled Bath) | 60° C. (140° F.) | |
| Gas | Compressed Air | |
| Gas flow rate(s) | 1,000 | mL/min |

| | |
|---|---|
| Sparge Material | Steel |

Because the test conditions for Example B involve higher temperature than an ambient room temperature, Example B is performed in a system 200 as illustrated in FIG. 5, where the system 200 includes a temperature-controlled bath apparatus 201.

The test for Example B can be run similarly as described for Example A. Data over time can be obtained like for the data shown in Table A2 for Example A. Table B2 shows the data for Example B.

TABLE B2
Example A Test Data

| | | | Liquid Vol. | Total | | Calc. Liq. | Calc. | Calc. |
|---|---|---|---|---|---|---|---|---|
| | | | (mL) (aka | Vol. of | | (%) not in | Liq. | Avg. |
| | | Delta | “Liq. | Liquid | Foam | Foam (aka | (mL) | Foam |
| | Time | Time | Hold Up | and Foam | Vol. | “Hold Up | in | Dens |
| Event | (min:sec) | (sec) | (mL)”) | (mL) | (mL) | Retained %”) | Foam | (g/L) |
| Gas on | 59:19.0 | 0.00 | 200 | 200 | 0 | 100 | 0 | N/A |
| | 59:20.6 | 1.56 | 190 | 450 | 260 | 95 | 5 | 44.7 |
| | 59:22.1 | 3.12 | 180 | 470 | 290 | 90 | 10 | 80.2 |
| | 59:23.7 | 4.68 | 170 | 530 | 360 | 85 | 15 | 96.9 |
| | 59:25.2 | 6.24 | 160 | 560 | 400 | 80 | 20 | 116.3 |
| | 59:26.8 | 7.80 | 150 | 600 | 450 | 75 | 25 | 129.2 |
| | 59:28.4 | 9.36 | 140 | 655 | 515 | 70 | 30 | 135.5 |
| | 59:30.0 | 10.96 | 130 | 700 | 570 | 65 | 35 | 142.8 |
| | 59:31.5 | 12.52 | 120 | 740 | 620 | 60 | 40 | 150.1 |
| | 59:33.1 | 14.08 | 110 | 790 | 680 | 55 | 45 | 153.9 |
| | 59:34.6 | 15.64 | 100 | 840 | 740 | 50 | 50 | 157.2 |
| | 59:36.2 | 17.20 | 90 | 910 | 820 | 45 | 55 | 156.0 |
| | 59:37.8 | 18.76 | 80 | 990 | 910 | 40 | 60 | 153.4 |
| | 59:39.3 | 20.32 | 70 | 1000 | 930 | 35 | 65 | 162.6 |
| | 59:40.9 | 21.88 | 60 | 1000 | 940 | 30 | 70 | 173.2 |
| | 59:42.4 | 23.44 | 49 | 1000 | 951 | 25 | 75 | 184.7 |
| Gas off | 59:44.0 | 25.00 | 38 | 1000 | 962 | 19 | 81 | 195.8 |
| | 00:00.0 | 41.00 | 50 | 990 | 940 | 25 | 75 | 185.6 |
| | 00:10.0 | 51.00 | 60 | 980 | 920 | 30 | 70 | 177.0 |
| | 00:20.0 | 61.00 | 70 | 960 | 890 | 35 | 65 | 169.9 |
| | 00:30.0 | 71.00 | 80 | 955 | 875 | 40 | 60 | 159.5 |
| | 00:39.0 | 80.00 | 90 | 950 | 860 | 45 | 55 | 148.8 |
| | 00:49.0 | 90.00 | 100 | 950 | 850 | 50 | 50 | 136.8 |
| | 00:58.0 | 99.00 | 110 | 950 | 840 | 55 | 45 | 124.6 |
| | 01:07.0 | 108.00 | 120 | 950 | 830 | 60 | 40 | 112.1 |
| | 01:18.0 | 119.00 | 130 | 940 | 810 | 65 | 35 | 100.5 |
| | 01:29.0 | 130.00 | 140 | 935 | 795 | 70 | 30 | 87.8 |
| | 01:51.0 | 152.00 | 150 | 930 | 780 | 75 | 25 | 74.6 |
| | 02:11.0 | 172.00 | 160 | 915 | 755 | 80 | 20 | 61.6 |
| | 02:41.0 | 202.00 | 170 | 915 | 745 | 85 | 15 | 46.8 |
| | 03:19.0 | 240.00 | 180 | 915 | 735 | 90 | 10 | 31.6 |
| | 04:13.0 | 294.00 | 190 | 890 | 700 | 95 | 5 | 16.6 |
| End | 05:25.0 | 366.00 | 198 | 915 | 717 | 99 | 1 | 3.2 |

Table B3 summarizes the data and evaluation boundaries and the occurrences that can be determined from the measurements for Example B.

TABLE B3
Example B Evaluation Boundaries

| | | Liquid Vol. | Total | | Liq. | |
|---|---|---|---|---|---|---|
| | | (mL) (aka | Vol. of | | Vol. | Avg. |
| | Delta | “Liq. | Liquid | Foam | (%) | Foam |
| | Time | Hold Up | and Foam | Vol. | not in | Density |
| Event | (sec) | (mL)”) | (mL) | (mL) | foam | (g/L) |
| Gas On | 0 | 200 | 200 | 0 | 100 | N/A |
| Gas Off | 25 | 38 | 1000 | 962 | 19 | 195.8 |
| End | 366 | 198 | 915 | 717 | 99 | 3.2 |
| t(½) Liquid Volume (%) | 107 | 119 | N/A | N/A | 59.5 | N/A |
| t(½) Foam Volume (mL) | N/A | N/A | N/A | 481 | N/A | N/A |
| t(½) Avg. Foam Density (g/L) | 118 | N/A | N/A | N/A | N/A | 97.9 |

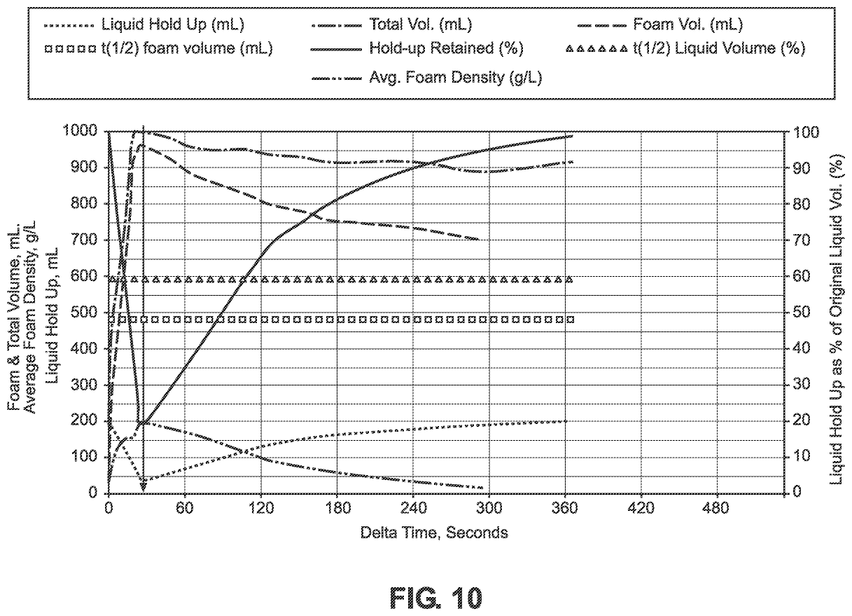

FIG. 10 is a graphical presentation of the data from Example B and Table B2.

As can be determined graphically from FIG. 10, the “t(½) Liquid Volume (%)” of 59.5% for Example B occurs at 107 seconds, which is 82 seconds after gas off time of 25 seconds. Therefore, in this Example B the t(½) Liquid Volume (%) occurs at 82 seconds after gas off.

As can be determined graphically from FIG. 10, the “t(½) Foam Volume (mL)” of 481 mL for Example B does not ever occur at all during the test. By conventional measurement standards, this would have indicated a good foamer result.

As can be determined graphically from FIG. 10, the t(½) Foam Density (g/L) of 97.9 g/L for Example B occurs at 118 seconds, which is 93 seconds after gas off time of 25 seconds. Therefore, the t(½) Foam Density (g/L) occurs at 93 seconds after gas off.

### Example C—Foamer Y in Water

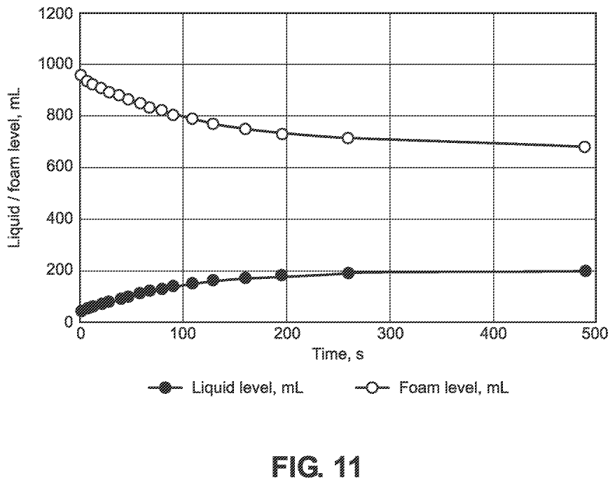

Example C illustrates a difference between the information obtained from focusing on the foam as compared to a new improved method focusing on the liquid is illustrated graphically in FIG. 11. In this example, water with Foamer Y at a concentration of 5 gallon per thousand is used with gas sparging to create a foam in a graduated cylinder, in a system 200 with a temperature-controlled bath apparatus 201 as illustrated in FIG. 5. Table C1 summarizes the test conditions used in this Example C.

TABLE C1
Example C Test Conditions

| Total Fluid Volume | 200 | mL |
|---|---|---|
| Aqueous phase | Sample of a Produced Water | |
| Fraction | 100% | |
| Volume | 200 | mL |
| TDS (Total Dissolved Solids) | N/A | |
| SG (Specific Gravity) | 1.163 | |
| Hydrocarbon phase | None | |
| Fraction | 0% | |
| Volume | 0 | mL |
| SG (Specific Gravity) | N/A | |
| API | N/A | |
| Foamer | Foamer Y | |
| Concentration | 5 | gpt |
| Concentration fraction of volume | Total | |
| Volume | 2 | mL |
| Temperature (Controlled Bath) | 60° C. (140° F.) | |
| Gas | Compressed Air | |
| Gas flow rate(s) | 1,000 | mL/min |

| | |
|---|---|
| Sparge Material | Steel |

FIG. 11 is a graphical comparison of an example of the difference between measurements of the decreasing foam volume (mL) over time vs the liquid volume (mL) dropping out of a foam over time, where FIG. 11 shows data after stopping gas flow.

Referring to FIG. 11, the data obtained from evaluating a foamer is illustrated as the upper plotted line for foam level, mL where this upper plotted line is the volume of the foam in the graduated cylinder over time. According to this information, a foam is created and measured for how fast the level of the foam drops in a graduated cylinder. The measured volume in the graduated cylinder is the total volume of both the foam and liquid because the liquid is recovering at the bottom. In this example, a foam is created with 200 mL of liquid plus gas to foam the liquid, such that the total volume of foam and liquid is initially about 950 mL as shown in FIG. 11. Then, over time, the total volume of foam and liquid remains stable at above 600 mL for nearly 500 seconds. This may appear as a good foam because the total volume of foam and liquid, which is mostly foam volume, is decreasing slowly and after nearly 10 minutes about 650 mL, which is about 70% of the initial total volume of total foam and liquid, is remaining in the graduated cylinder. So, according to the measuring of the total volume of foam and liquid volume in the graduated cylinder, this would indicate a good, stable foam.

However, continuing to refer to FIG. 11, the reality is the lower plotted line for liquid level, mL, where this lower plotted line is the volume of the liquid in the bottom of the graduated cylinder over time. For the same foam, the volume of liquid is initially only about 25 mL at the bottom of the graduated cylinder, where the foam is nearly 100% of the total volume of foam and liquid in the graduated cylinder. But in only about the first 2 minutes, about 80% of the initial liquid used to create the foam is recovered at the bottom of the graduated cylinder. In about the first 4 minutes, nearly all the liquid has fallen out of the foam. Accordingly, the average density of the foam rapidly decreases as the liquid falls out of the foam volume.

Where focusing on the total volume of foam and liquid would indicate this Foamer Y at 5 gpt concentration produces a stable foam, the reality as illustrated in FIG. 11 according to this improved method of foamer analysis shows that, no, the liquid is falling out of the foam at a high rate. This example provided only about 2 minutes of liquid stability in the foam. The liquid volume of 200 mL is about 90% in the foam when initially foamed with gas where only about 25 mL of the liquid at the bottom of the graduated cylinder. Then, most of the liquid dropped out of the foam to the bottom and recovered back up to 190 mL in less than 2 minutes, so, this is not a satisfactory performance of the foamer. The reality is liquid is falling out of that foam at quite high rate. In this Example C, there is a quite low concentration of 5 gallon of foamer per thousand gallons of liquid and a low gas flow rate, so, the recommendation here in this example is to change the concentration of the foamer and test at the different concentration.

In preferred embodiments, as a rule of thumb, stability of liquid in the foam is preferably in a range of 5-6 minutes.

Only measuring the change over time for the total height of the foam and liquid in a graduated cylinder as illustrated in FIG. 11 for the total foam and liquid volume would not provide a good evaluation of the foam stability. In the improved system and methods, it is recognized that the foam height can remain high even as most of the liquid falls out from foam. The density of the foam decreases, and in some cases the density of the foam decreases so much that the opacity of the foam decreases so much that it may be possible to see through the foam.

### Example D—Foamer Y in 40% Synthetic Water

An example of another test of the water-based (hydrophilic) Foamer Y is provided in Example D. In this Example D, the Foamer Y is tested with 40% synthetic water and with 60% hydrocarbon liquid (v/v). In this example, the Foamer Y at a concentration of 10 gallon per thousand is used with gas sparging to create a foam in a graduated cylinder, in a system 200 with a temperature-controlled bath apparatus 201 as illustrated in FIG. 5. Table D1 summarizes the test conditions used in this Example D.

TABLE D1
Example D Test Conditions

| Total Fluid Volume | 200 | mL |
|---|---|---|
| Aqueous phase | Synthetic Water | |
| Fraction | 40% | |
| Volume | 80 | mL |
| TDS (Total Dissolved Solids) | 2,215 | |
| SG (Specific Gravity) | 1.163 | |
| Hydrocarbon phase | Kerosene (analytical grade) | |
| Fraction | 60% | |
| Volume | 120 | mL |
| SG (Specific Gravity) | 0.8 | |
| API | N/A | |
| Foamer | Foamer Y | |
| Concentration | 10 | gpt |
| Concentration fraction of volume | Total | |
| Volume | 2 | mL |
| Temperature (Controlled Bath) | 50° C. (122° F.) | |
| Gas | Compressed Air | |
| Gas flow rate(s) | 1,000 | mL/min |

| | |
|---|---|
| Sparge material | Steel |

Because the test conditions for Example D involve higher temperature than an ambient room temperature, Example D is performed in a system 200 as illustrated in FIG. 5, where the system 200 includes a temperature-controlled bath apparatus 201.

An example of a foam density profile over time cab be obtained as shown in Table D2, which is calculated as described for Table A2.

TABLE D2
Example D Test Data

| | | | Liquid Vol. | Total | | Calc. Liq. | Calc. | Calc. |
|---|---|---|---|---|---|---|---|---|
| | | | (mL) (aka | Vol. of | | (%) not in | Liq. | Avg. |
| | | Delta | “Liq. | Liquid | Foam | Foam (aka | (mL) | Foam |
| | Time | Time | Hold Up | and Foam | Vol. | “Hold Up | in | Dens |
| Event | (min:sec) | (sec) | (mL)) | (mL) | (mL) | Retained %”) | Foam | (g/L) |
| Gas on | 42:33.0 | 0.00 | 200 | 200 | 0 | 100 | 0 | N/A |
| | 42:35.0 | 2.00 | 190 | 300 | 110 | 95 | 10 | 105.7 |
| | 42:37.0 | 4.00 | 180 | 320 | 140 | 90 | 20 | 166.1 |
| | 42:39.0 | 6.00 | 170 | 340 | 170 | 85 | 30 | 205.2 |
| | 42:42.0 | 9.00 | 160 | 560 | 400 | 80 | 40 | 116.3 |
| | 42:45.0 | 12.00 | 150 | 600 | 450 | 75 | 50 | 129.2 |
| | 42:48.0 | 15.00 | 140 | 655 | 515 | 70 | 60 | 135.5 |
| | 43:08.0 | 35.00 | 130 | 700 | 570 | 65 | 70 | 142.8 |
| | 43:14.0 | 41.00 | 120 | 740 | 620 | 60 | 80 | 150.1 |
| | 43:16.0 | 43.00 | 110 | 800 | 690 | 55 | 90 | 151.7 |
| | 43:20.0 | 47.00 | 110 | 840 | 730 | 55 | 90 | 143.4 |
| | 43:24.0 | 51.00 | 110 | 910 | 800 | 55 | 90 | 130.8 |
| | 43:31.0 | 58.00 | 110 | 990 | 880 | 55 | 90 | 118.9 |
| | 43:33.0 | 60.00 | 110 | 1000 | 890 | 55 | 90 | 117.6 |
| | 43:34.0 | 61.00 | 110 | 1000 | 890 | 55 | 90 | 117.6 |
| | 43:35.0 | 62.00 | 110 | 1000 | 890 | 55 | 90 | 117.6 |
| Gas off | 43:43.0 | 70.00 | 110 | 1000 | 890 | 55 | 90 | 117.6 |
| | 45:08.0 | 155.00 | 120 | 980 | 860 | 60 | 80 | 108.2 |
| | 45:38.0 | 185.00 | 130 | 980 | 850 | 65 | 70 | 95.8 |
| | 45:50.0 | 197.00 | 135 | 940 | 805 | 68 | 65 | 93.9 |
| | 46:02.0 | 209.00 | 140 | 900 | 760 | 70 | 60 | 91.8 |
| | 46:27.0 | 234.00 | 150 | 860 | 710 | 75 | 50 | 81.9 |
| | 46:58.0 | 265.00 | 160 | 800 | 640 | 80 | 40 | 72.7 |
| | 47:20.0 | 287.00 | 165 | 750 | 585 | 83 | 35 | 69.6 |
| | 48:00.0 | 327.00 | 170 | 650 | 480 | 85 | 30 | 72.7 |
| | 48:55.0 | 382.00 | 180 | 550 | 370 | 90 | 20 | 62.9 |
| | 49:39.0 | 426.00 | 190 | 450 | 260 | 95 | 10 | 44.7 |
| | 50:02.0 | 449.00 | 190 | 400 | 210 | 95 | 10 | 55.4 |
| | 50:37.0 | 484.00 | 190 | 350 | 160 | 95 | 5 | 72.7 |
| | 51:11.0 | 518.00 | 195 | 300 | 105 | 98 | 2 | 55.4 |
| | 51:50.0 | 557.00 | 195 | 250 | 55 | 98 | 2 | 105.7 |
| End | 52:14.0 | 581.00 | 195 | 200 | 5 | 98 | 2 | 1163.0 |

Table D3 summarizes the data and evaluation boundaries and the occurrences that can be determined from the measurements for Example D.

TABLE D3
Example D Evaluation Boundaries

| | | Liquid Vol. | Total | | Liq. | |
|---|---|---|---|---|---|---|
| | | (mL) (aka | Vol. of | | Vol. | Avg. |
| | Delta | “Liq. | Liquid | Foam | (%) | Foam |
| | Time | Hold Up | and Foam | Vol. | not in | Density |
| Event | (sec) | (mL)”) | (mL) | (mL) | foam | (g/L) |
| Gas On | 0 | 200 | 200 | 0 | 100 | N/A |
| Gas Off | 70 | 110 | 1000 | 890 | 55 | 117.6 |
| End | 581 | 195 | 200 | 5 | 97.5 | 1163.0 |
| t(½) Liquid Volume (%) | 243 | 155 | N/A | N/A | 77.5 | N/A |
| t(½) Foam Volume (mL) | 345 | N/A | N/A | 445 | N/A | N/A |
| t(½) Avg. Foam Density (g/L) | 148 | N/A | N/A | N/A | N/A | 58.8 |

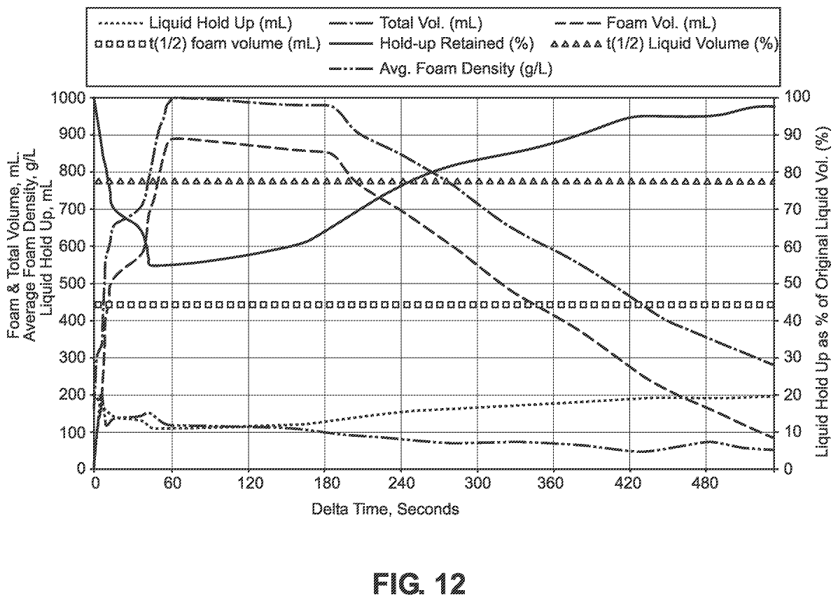

FIG. 12 is a graphical presentation of the data from Example D and Table D3.

As can be determined graphically from FIG. 12, the “t(½) Liquid Volume (%)” of 5% for Example D occurs at 243 seconds, which is 173 seconds after gas off time of 70 seconds.

As can be determined graphically from FIG. 12, the “t(½) Foam Volume (mL)” of 445 mL for Example D occurs at about 345 seconds, which is about 275 seconds after gas off time of 70 seconds. However, this Example D is a test designed for produced water unloading. The focus is the liquid column at the bottom of the graduated cylinder and the rate at which liquid recovers. We measure how long it takes for half of the liquid that is foamed to be returned back into the liquid column at the bottom of the graduated cylinder. This is a critical measurement of interest. The moving the liquid out of the well and how fast the liquid folds back into the well after stopping gas injection is a real indication of my foam stability. This is in contrast to the conventional method that is based on foam height, which would yield misleading results in some cases where little or nothing happens to total foam height after all the liquid is recovered, where the foam density is decreased, and it is possible to see right through the foam.

As can be determined graphically from FIG. 12, the “t(½) Foam Density (g/L)” of 58.8 g/L for Example D occurs at 148 seconds, which is 78 seconds after gas off time of 70 seconds.

### Example E—“Blank” (No Foamer) with Only a Hydrocarbon Liquid

Example E is a test of a “blank” without any foamer in a condensate (100% kerosene). In this example, gas sparging is used to create a foam in a graduated cylinder, in a system 200 with a temperature-controlled bath apparatus 201 as illustrated in FIG. 5. Table E1 summarizes the test conditions used in this Example E.

TABLE E1
Example E Test Conditions

| | |
|---|---|
| Total Fluid Volume | 200 mL |
| Aqueous phase | None |
| Fraction | 0% |
| Volume | 0 mL |
| TDS (Total Dissolved Solids) | N/A |
| SG (Specific Gravity) | N/A |
| Hydrocarbon phase | Kerosene (analytical grade) |
| Fraction | 100% |
| Volume | 200 mL |
| SG (Specific Gravity) | 0.8 |
| API | N/A |
| Foamer | None |
| Concentration | N/A |
| Concentration fraction of volume | N/A |
| Volume | N/A |
| Temperature (Controlled Bath) | 50° C. (122° F.) |
| Gas | Nitrogen |
| Gas flow rate(s) | 5, 7, 10, 12, and 14 L/min |
| Sparge material | Aluminum |

Because the test conditions for Example E involve higher temperature than an ambient room temperature, Example E is performed in a system 200 as illustrated in FIG. 5, where the system 200 includes a temperature-controlled bath apparatus 201.

Table E2 shows a test log summary of the data for Example E.

TABLE E2
Example E Test Log Summary

| | | | Total | | |
|---|---|---|---|---|---|
| | | Gas | Liquid | | |
| | | Flow | & Foam | Liquid | Volume |
| Time | | Rate | Volume | Volume | Unloaded |
| (min:sec) | Event | (L/min) | (mL) | (mL) | (mL) |
| 0:33 | Start gas flow | 5 | 200 | 200 | 0 |
| 0:45 | Equilibrium | 5 | 300 | 140 | 0 |
| 1:00 | Increase gas | 7 | N/A | N/A | 0 |
| | flow rate | | | | |
| 1:14 | Equilibrium | 7 | 400 | 120 | 0 |
| 1:38 | Increase gas | 10 | N/A | N/A | 0 |
| | flow rate | | | | |
| 1:44 | Equilibrium | 10 | 500 | 100 | 0 |
| 2:05 | Increase | 12 | N/A | N/A | 0 |
| | flow rate | | | | |
| 2:21 | Equilibrium | 12 | 750 | 65 | 0 |
| 2:40 | Increase gas | 14 | N/A | N/A | 0 |
| | flow rate | | | | |
| 2:51 | Equilibrium | 14 | 1000 | 50 | 0 |
| 3:29 | Stop gas flow | 0 | 1000 | 50 | 0 |
| 3:36 | Liquid | 0 | 200 | 200 | 0 |
| | recovered | | | | |

This test is carried out at gas flow rates up to 14 L/min with no hydrocarbon (in this case, kerosene) being foamed out of the first graduated cylinder 204. At 14 L/min, the foam being generated and refluxed reached equilibrium with a total foam and liquid volume of 1,000 mL. It is noted that the nitrogen gas is entering the fluid from the very bottom of the frit for sparging, but the minimum liquid level recorded is 50 mL. Once the gas flow is turned off, the total liquid volume is recovered extremely quickly, in about 7 seconds. As illustrated by this Example E, the kerosene, without any foamer added, has some minor foaming tendency at high gas flow rates. The foam generated is a function of gas flow rate. In this Example E, the foam column is very unstable and collapses as soon as the gas flow stops.

### Example F—Foamer Z at 2% v/v in Only Hydrocarbon

An example of a test of a hydrocarbon-based Foamer Z at 2% v/v (or 20 gpt) concentration in hydrocarbon is provided in Example F. In this Example F, the Foamer Z is tested in a system 300 with a temperature-controlled bath apparatus 201 and gas sparging to create a foam in a first graduated cylinder 204 operatively connected to collect the foam in a second graduated cylinder 342 as illustrated in FIG. 6.

In this Example F, the system 300 can be used to measure the volume or mass of fluid being carried out of the first graduated cylinder 204 at sufficiently high gas flow rates into the second graduated cylinder 342. In various embodiments, the mass measurements can be manually recorded over time. In the alternative, a system 400 as illustrated in FIG. 7 can be used so that that mass measurements can be automatically recorded over time.

Table F1 summarizes the test conditions used in this Example F.

TABLE F1
Example F Test Conditions

| Total Fluid Volume | 200 | mL |
|---|---|---|
| Aqueous phase | None | |
| Fraction | 0% | |
| Volume | 0 | mL |
| TDS (Total Dissolved Solids) | N/A | |
| SG (Specific Gravity) | N/A | |
| Hydrocarbon phase | Kerosene (analytical grade) | |
| Fraction | 100% | |
| Volume | 200 | mL |
| SG (Specific Gravity) | 0.8 | |
| API | N/A | |
| Foamer | Foamer Z | |
| Concentration | 2% | v/v |
| Concentration fraction of volume | 4 | mL |

| | |
|---|---|
| Volume | N/A |
| Temperature (Controlled Bath) | 50° C. (122° F.) |
| Gas | Nitrogen |
| Gas flow rate(s) | 5, 7, and 10 L/min |
| Sparge material | Aluminum |

Table F2 shows a test log summary of the data for Example F.

TABLE F2
Example F Test Log Summary

| | | | | | Volume |
|---|---|---|---|---|---|
| | | | | | Unloaded (mL) |
| | | | Total Liquid | | (based on SG |
| | | | & Foam | Liquid | and measured |
| | | Gas | Volume (mL) | Volume (mL) | in second |
| | | Flow | (in first | (in first | graduated |
| Time | | Rate | graduated | graduated | cylinder on |
| (seconds) | Event | (L/min) | cylinder) | cylinder) | mass balance) |
| 25 | Start gas flow | 5 | 205 | 205 | 0 |
| 41 | Initial foam reaches top of | 5 | 1000 | 25 | 0 |
| | the first graduated cylinder | | | | |
| 51 | Foam exiting the first | 5 | >1000 | 25 | 0 |
| | graduated cylinder | | | | |
| 88 | Start to get returns | 5 | >1000 | N/A | 0 |
| | at the mass balance | | | | |
| 177 | Full column of the first | 5 | >1000 | 0 | 22 |
| | graduated cylinder is foam | | | | |
| 331 | Increase gas flow rate | 7 | >1000 | N/A | 94 |
| 368 | Increase gas flow rate | 10 | >1000 | N/A | 102 |
| 488 | Stop gas flow | 0 | >1000 | 0 | 127 |
| 523 | Liquid recovered in the | 0 | 50 | 50 | N/A |
| | first graduated cylinder | | | | |
| 562 | End recording | 0 | 50 | 50 | N/A |

In this Example F, the Foamer Z is at a concentration of 2% by volume or 20 gpt. The test is started at an initial nitrogen gas flow rate of 5 L/min. The gas is observed entering the fluid from the bottom of the sparge, that is, at the bottom of the delivery tube 216 and frit 220 illustrated in FIG. 6. In this Example F, a gas flow rate of 5 L/min is sufficient to quickly generate a foam flow upward in the first graduated cylinder 204, out and through the third gas tubing 346, and into the second graduated cylinder 342 on the mass balance 340.

At time 41 seconds, that is, 16 seconds after starting the gas flow at a gas flow rate of 5 L/min, the foam reaches the top of the first graduated cylinder 204. As the foam reaches the top of the first graduated cylinder 204, the liquid column in the first graduated cylinder has been reduced to 25 mL, indicating that a total of 175 mL of liquid is foamed in the initial 16 seconds after starting the gas flow.

At time 51 seconds, that is, 26 seconds after starting the gas flow, the foam reaches the second graduated cylinder 342 on the mas balance 340. The liquid column in the first graduated cylinder has still been reduced to 25 mL, indicating that a total of about 174 mL of liquid is foamed in the initial 26 seconds after starting the gas flow.

At time 88 seconds, that is, 33 seconds after starting the gas flow, the mass balance 340 starts to show the collection of foam and liquid in the second graduated cylinder 342.

At time 331 seconds, that is, at 5:31 minutes, the gas flow rate is increased to 7 L/min and observations are continued to be made.

At time 368 seconds, that is, 6:08 minutes, the gas flow rate is increased to 10 L/min.

In Example F, as the liquid continues to be foamed and carried out from the liquid column, the foam density inside the second graduated cylinder 204 decreases and a change in flow pattern from a continuous annular-mist flow pattern to a slug flow pattern could be observed and recorded in terms of liquid delivery rate to the second graduated cylinder 342 (at constant gas flow rate).

Once the gas flow is turned off at time 488 seconds, that is, at 8:08 minutes, in this Example F, the liquid recovery rate is much slower than in the case of the blank test of Example E. In this Example F, the rarified foam remaining in the first graduated cylinder 204 when the gas flow rate is stopped fully collapsed in 35 seconds, 50 mL of the original liquid column of 200 mL remained in the first graduated cylinder 204 at the end of the test indicating about 75% removal of hydrocarbon from the first graduated cylinder.

According to an embodiment, graphical plots of the volume or mass of a discharged fluid (foam or the liquid in the foam) vs time at specified gas flow rate can be used to evaluate and compare foamers.

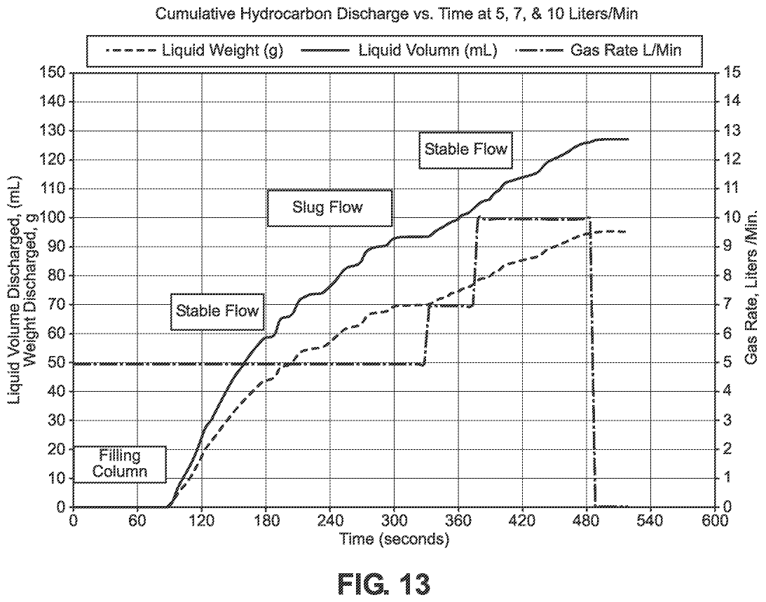

FIG. 13 is a graphical presentation of the data from Example F, including as summarized in Table F2, where FIG. 13 shows the gas flow rates over time and shows the liquid volume (mL) of the liquid collapsed from the foam and the liquid weight (g) of the collapsed liquid from the foam as collected in the second graduated cylinder 342 over time.

From the plot of the cumulative volume of fluid discharged into the second graduated cylinder 342 vs time as shown in FIG. 13, it is seen that the rate of discharge at constant gas flow rate is constantly decreasing.

The average foam density in the foam column in the first graduated cylinder 204 is also decreasing with time as the fluid is removed from the column. As indicated on FIG. 13, as the foam density decreases, the flow pattern at constant gas flow rate changes from a continuous annular-mist flow pattern to a slug flow pattern. This phenomenon is recorded on the plot of fluid volume discharges vs. time and can also be observed on a video of the first graduated cylinder 204. About 87% of the hydrocarbons are in a two-phase foam flow within 16 seconds of starting the gas flow at 5 L/min.

Accordingly, Foamer Z at 2% concentration appears to effectively generate foam with kerosene.

### Example G—Foamer Z at 4% v/v in Only Hydrocarbon

An example of a test of the hydrocarbon-based Foamer Z at a higher concentration of 4% is provided in Example G. In this Example G, the Foamer Z is tested in a system 300 with a temperature-controlled bath apparatus 201 and gas sparging to create a foam in a first graduated cylinder 204 operatively connected to collect the foam in a second graduated cylinder 342 as illustrated in FIG. 6.

In this Example G, the system 300 can be used to measure the volume or mass of fluid being carried out of the first graduated cylinder 204 at sufficiently high gas flow rates into the second graduated cylinder 342. In various embodiments, the mass measurements can be manually recorded over time. In the alternative, a system 400 as illustrated in FIG. 7 can be used so that that mass measurements can be automatically recorded over time.

Table G1 summarizes the test conditions used in this Example G.

TABLE G1
Example F Test Conditions

| Total Fluid Volume | 200 | mL |
|---|---|---|
| Aqueous phase | None | |
| Fraction | 0% | |
| Volume | 0 | mL |
| TDS (Total Dissolved Solids) | N/A | |
| SG (Specific Gravity) | N/A | |
| Hydrocarbon phase | Kerosene (analytical grade) | |
| Fraction | 100% | |
| Volume | 200 | mL |
| SG (Specific Gravity) | 0.8 | |
| API | N/A | |
| Foamer | Foamer Z | |
| Concentration | 4% | v/v |
| Concentration fraction of volume | 8 | mL |

| | |
|---|---|
| Volume | N/A |
| Temperature (Controlled Bath) | 50° C. (122° F.) |
| Gas | Nitrogen |
| Gas flow rate(s) | 5, 7, and 10 L/min |
| Sparge material | Aluminum |

Table G2 shows a test log summary of the data for Example G.

TABLE G2
Example G Test Log Summary

| | | | | | Volume |
|---|---|---|---|---|---|
| | | | | | Unloaded (mL) |
| | | | Total Liquid | | (based on SG |
| | | | & Foam | Liquid | and measured |
| | | Gas | Volume (mL) | Volume (mL) | in second |
| | | Flow | (in first | (in first | graduated |
| Time | | Rate | graduated | graduated | cylinder on |
| (seconds) | Event | (L/min) | cylinder) | cylinder) | mass balance) |
| 0 | Start gas glow | 5 | 220 | 220 | 0 |
| 15 | Initial foam reaches | 5 | 1000 | 25 | 0 |
| | top of the first | | | | |
| | graduated cylinder | | | | |
| 22 | Foam exiting the first | 5 | >1000 | 20 | 0 |
| | graduated cylinder | | | | |
| 66 | Start to get returns | 5 | >1000 | 20 | 0 |
| | at the balance | | | | |
| 85 | N/A | 5 | >1000 | 5 | 20 |
| 120 | N/A | 5 | >1000 | 5 | 51 |
| 200 | Full column in the first | 5 | >1000 | 5 | 90 |
| | graduated cylinder is | | | | |
| | essentially all foam | | | | |
| 240 | Increase gas flow rate | 7 | >1000 | 0 | 100 |
| 540 | Stop gas flow | 0 | >1000 | 0 | 150 |
| 583 | Liquid in the first | 0 | 50 | 50 | 150 |
| | graduated cylinder | | | | |
| | recovered | | | | |
| 593 | End recording | N/A | N/A | N/A | N/A |

In Example G, the Foamer Z is at a concentration of 4% by volume or 40 gpt. The test is started at an initial nitrogen gas flow rate of 5 L/min. The gas is observed entering the fluid from the bottom of the sparge, that is, at the bottom of the delivery tube 216 and frit 220 illustrated in FIG. 6. In this Example G, a gas flow rate of 5 L/min is sufficient to quickly generate a foam flow upward in the first graduated cylinder 204, out and through the third gas tubing 346, and into the second graduated cylinder 342 on the mass balance 340.

At time 15 seconds after starting the gas flow at a gas flow rate of 5 L/min, the foam reaches the top of the first graduated cylinder 204. As the foam reaches the top of the first graduated cylinder 204, the liquid column in the first graduated cylinder has been reduced to 25 mL, indicating that a total of about 195 mL of liquid is foamed in the initial 15 seconds after starting the gas flow.

At time 22 seconds after starting the gas flow, the foam reaches the second graduated cylinder 342 on the mas balance 340. The liquid column in the first graduated cylinder has been reduced to 20 mL, indicating that a total of about 200 mL of liquid is foamed in the initial 22 seconds after starting the gas flow.

At time 66 seconds after starting the gas flow, the mass balance 340 starts to show the collection of foam and liquid in the second graduated cylinder 342.

At time 240 seconds, that is, at 4:00 minutes, the gas flow rate is increased to 7 L/min and observations are continued to be made.

In Example G, as the liquid continues to be foamed and carried out from the liquid column, the foam density inside the second graduated cylinder 204 decreases and a change in flow pattern from a continuous annular-mist flow pattern to a slug flow pattern could be observed and recorded in terms of liquid delivery rate to the second graduated cylinder 342 (at constant gas flow rate).

Once the gas flow is turned off at time 540 seconds, that is, at 9:00 minutes, in this Example G, the liquid recovery rate is much slower than in the case of the blank test of Example E. In this Example G, the rarified foam column remaining in the first graduated cylinder 204 when the gas flow rate is stopped fully collapsed in 43 seconds, 50 mL of the original liquid column of 220 mL remained in the first graduated cylinder 204 at the end of the test indicating about 77% removal of hydrocarbon from the first graduated cylinder.

According to an embodiment, graphical plots of the volume or mass of a discharged fluid (foam or the liquid in the foam) vs time at specified gas flow rate can be used to evaluate and compare foamers.

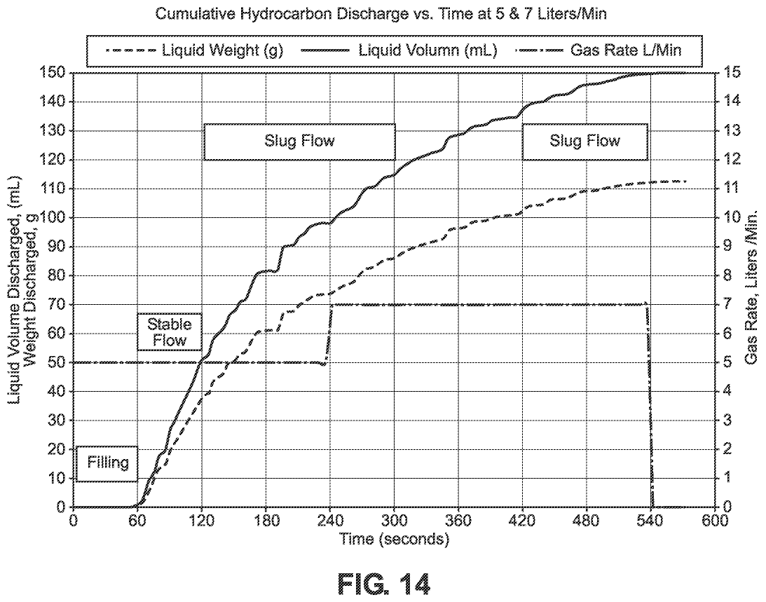

FIG. 14 is a graphical presentation of the data from Example G, including as summarized in Table G2, where FIG. 14 shows the gas flow rates over time and shows the liquid volume (mL) of the liquid collapsed from the foam and the liquid weight (g) of the collapsed liquid from the foam as collected in the second graduated cylinder 342 over time.

From the plot of the cumulative volume of fluid discharged into the second graduated cylinder 342 vs time as shown in FIG. 14, it is seen that the rate of discharge at constant gas flow rate is constantly decreasing.

The average foam density in the foam column in the first graduated cylinder 204 is also decreasing with time as the fluid is removed from the column. As indicated on FIG. 14, as the foam density decreases, the flow pattern at constant gas flow rate changes from a continuous annular-mist flow pattern to a slug flow pattern. This phenomenon is recorded on the plot of fluid volume discharges vs. time and can also be observed on a video of the first graduated cylinder 204. About 89% of the hydrocarbons are in a two-phase foam flow within 15 seconds of starting the gas flow at 5 L/min.

Accordingly, Foamer Z at 4% concentration appears to effectively generate foam with kerosene.

### Example H—Comparison of Foamer Z at 2% and at 4% v/v in Hydrocarbon

From the comparison of discharge rates vs time for tests Example F and Example G, it can be determined that at 4% Foamer Z in Example G both the rate and the total quantity of hydrocarbon recovered in the second graduated cylinder 342 is greater.

At 4% v/v Foamer Z, the foam is generated faster and more liquid is recovered than in the test with 2% v/v Foamer Z. The initial foam quality also appeared to be better in terms of observed bubble size. It is not possible to realistically qualify foam stability as >80% of the liquid had been removed from the first graduated cylinder 204 at the end of the test.

In Example F, the final foam column collapses in about 35 seconds, about 50 mL of liquid is recovered in the first graduated cylinder 204 at the end of the test indicating about 150 mL from an initial 200 mL or 75% removal of hydrocarbon from the first graduated cylinder 204. In Example G, the final foam column collapses in about 35 seconds, about 50 mL of liquid is recovered in the first graduated cylinder 204 at the end of the test indicating about 170 mL from an initial 220 mL or 77% removal of hydrocarbon from the first graduated cylinder 204.

In conclusion, from a comparison of Example F and Example G, the Foamer Z performs better at foaming kerosene at 4% than at 2% concentration.

Improved Systems and Methods with a Condenser for Collecting Foam in a Second Vessel

As is evident from Example F and Example G, in some cases a portion of the liquid in the foam may be lost to the atmosphere as vapor instead of all being collected in the second graduated cylinder 342. According to another preferred embodiment, a condenser can be used to help collect the liquid for more accurate measurements and to reduce liquid in the foam from escaping to the atmosphere.

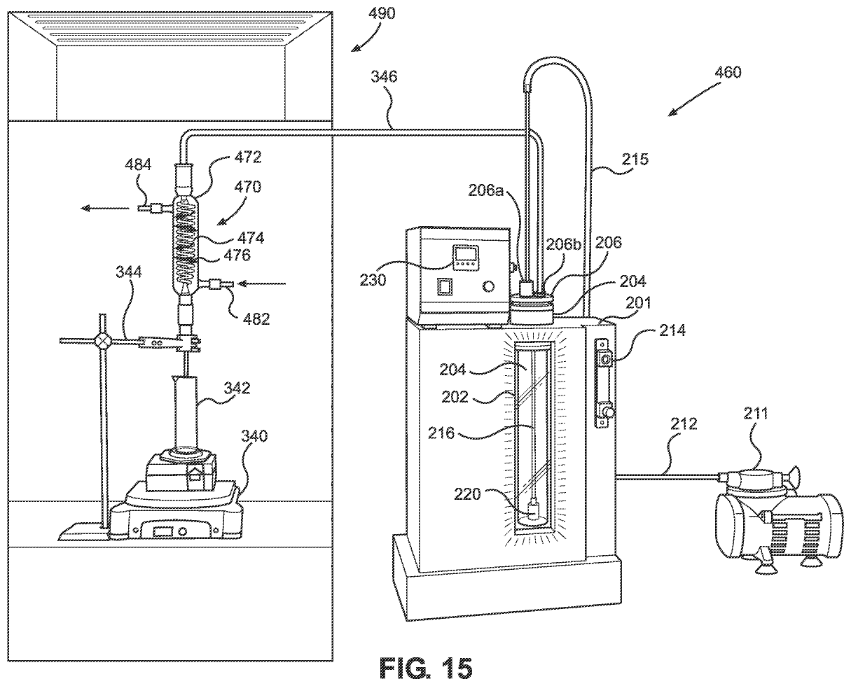

FIG. 15 is an illustration of another embodiment of a system according to the disclosure, wherein a system 460 that is like the system 300 illustrated in FIG. 6 (with the same referenced elements) additionally includes a condenser 470 (for example, of the Graham type) where the condenser 470 has an outer jacket 472, a spiral coil conduit 474 for fluid (e.g., gas, foam, vapor, or condensate) to flow from an upper end of the condenser 470 down through the spiral coil conduit 474 as indicated by arrows 476 to a lower end of the condenser 470, and a water flow path through the outer jacket 472 and around the spiral coil conduit 474, where the condenser 470 additionally has a lower water port 482 at the lower end of the condenser 470 into the jacket 472 and an upper water port 484 at an upper end of the condenser 470, whereby cool water (for example, tap water) can be flowed into either the lower water port 482 or the upper water port 484 (for example, into the lower water port 482) and out the other water port (for example, out the upper water port 484) at the other end of the condenser 470 around the spiral coil conduit 474 for cooling the spiral coil conduit 474, where foam and condensate can be collected from the lower end of the spiral coil conduit 474 of the condenser 470 into the second graduated cylinder 342 on the mass balance 340. Preferably, the mass balance 340, the second graduated cylinder 342, and the condenser 470 are positioned in a laboratory fume hood 490, where the fume hood 490 is to reduce exposure of laboratory technicians to potentially toxic vapors or gases.

The use of the condenser 470 helps cool the foam from a higher temperature that can be used in the temperature-controlled apparatus 201, which reduces loses of the liquid due to evaporation to the atmosphere.

### Further Exemplifications

The present disclosure may be further exemplified by the following numbered clauses and examples. 1. A system (300, 400) for evaluating a foamer for unloading liquid, the system comprising: (a) a temperature-controlled bath (202, 410) having a first vessel (204, 402) for containing a liquid in the first vessel (204, 402) at a temperature controlled by the temperature-controlled bath (202, 410) and for measuring the volume of a liquid or foam in the first vessel (204, 402); (b) a gas tubing (215, 412) operatively connected from a gas flow meter (214, 414) and a gas source (211, 411) to gas delivery tubing (216) and a frit (220) for sparging the liquid in the first vessel (204, 402) with a gas for making a foam; (c) a mass balance (340, 420); (d) a second vessel (342, 424) on the mass balance (340, 420); and (e) fluid tubing (346, 415) operatively connected from the first vessel (204, 402) in the temperature-controlled bath (202, 410) to the second vessel (342, 424). 2. The system (460) according to clause 1 or 2, additionally comprising a condenser (470) for the fluid tubing (346). 3. The system (400) according to clause 1, additionally comprising: (a) a camera (430) for recording changes in visual appearance of the foam in the first vessel (402) of the temperature-controlled bath (410); (b) a central processing unit (440) operatively connected to the mass balance (420) and to the camera (430) for recording and analyzing data from the mass balance (420) and from the camera (430); and (c) a user interface (450) operatively connected to the central processing unit (440).

The present disclosure may be further exemplified by the following numbered clauses and examples. 4. A method for evaluating a foamer, the method comprising the steps of: (a) combining (i) an aqueous phase, a hydrocarbon phase, or both an aqueous phase and a hydrocarbon phase in a predetermined proportion with (ii) a foamer to obtain a liquid, wherein the foamer is in a predetermined concentration in the liquid; (b) sparging the liquid with a gas under sparging conditions including a predetermined gas flow rate to create a foam from at least some of the liquid and at least some of the gas; and (c) during or after the step of sparging, determining the amount of the liquid in the foam, wherein the step of determining is performed one or more times. 5. The method according to clause 4, wherein the aqueous phase is water, a synthetic water composition simulating water obtained from a well, or a sample of water obtained from a well. 6. The method according to clause 4 or 5, wherein the hydrocarbon phase is kerosene, a hydrocarbon composition simulating a hydrocarbon obtained from a well, or a sample of a hydrocarbon obtained from a well. 7. The method according to clause 4, wherein the foamer is a hydrophilic foamer or an amphiphile foamer. 8. The method according to clause 4, wherein the gas is selected from the group consisting of air, nitrogen, carbon dioxide, or any combination thereof in any proportion. 9. The method according to clause 4, wherein the step of sparging additionally comprises controlling the temperature of the liquid during the step of sparging. 10. The method according to clause 4, wherein the step of determining the amount of the liquid in the foam comprises the steps: (a) measuring the volume of the liquid before the step of sparging; (b) during or after the step of sparging, measuring the volume of the liquid remaining that is not in the foam; and (c) subtracting the volume of liquid remaining that is not in the foam from the volume of the liquid before the step of sparging to determine a difference that is the amount of the liquid in the foam. 11. The method according to clause 4, wherein the step of determining the amount of the liquid in the foam comprises the steps: (a) recording the volume of the liquid before the step of sparging using a camera (430) operatively connected to a central processing unit (440); (b) over time during or after the step of sparging, recording the volume of the liquid remaining that is not in the foam using the camera (430) operatively connected to a central processing unit (440); and (c) analyzing the recorded volumes over time using the central processing unit (440) to determine the differences that are the volumes of the liquid in the foam over time. 12. The method according to clause 11, wherein recording the volume of the liquid is with a graduated cylinder (204) having graduated markings (204e). 13. The method according to clause 11, additionally comprising the step of graphically plotting the recorded volumes over time using the central processing unit (440) operatively connected to a user interface (450). 14. The method according to clause 4, additionally comprising the step of, over time during or after the step of sparging, recording with a camera (430) the visual appearance of the foam as it changes over time. 15. The method according to clause 4, wherein the step of determining the mass or the amount of the liquid in the foam comprises the steps of: (a) collecting the foam to obtain a collected foam and any of the liquid dropped from the collected foam; and (b) measuring the mass of the collected foam and any of the liquid dropped from the collected foam. 16. The method according to clause 15, wherein the step of collecting the foam additionally comprises cooling the foam to reduce any evaporation of the liquid in the foam to the atmosphere. 17. The method according to clause 16, wherein cooling the foam is with a condenser (470). 18. The method according to clause 16, additionally comprising the steps of: (a) over time during or after the step of sparging, recording the mass of the collected foam and any of the liquid dropped from the collected foam; and (b) analyzing the recorded masses using a central processing unit (440) to determine the amounts of the liquid in the collected foam over time. 19. The method according to clause 4, additionally comprising the steps of: (a) during or after the step of sparging, determining the volume of the foam; and (b) based on the step of determining the amount of the liquid in the foam, determining the density of the foam. 20. The method according to clause 4, wherein the steps of combining, sparging, and determining the amount of the liquid in the foam are steps of a discrete and separate test for the liquid and for the sparging conditions from another discrete and separate test for a different liquid or for different sparging conditions.

### Interpretation, Definitions, and Usages

### Basic Principles of Interpretation

The words, terms, phrases, and other symbols used herein have their plain, ordinary meaning to persons of skill in the art of this disclosure, except to the extent explicitly and clearly defined in this disclosure, on the condition that even if explicitly defined in this disclosure, the specific context of a usage could still require a different or more specific meaning. The definitions provided are intended to help clarify—not confuse or be applied blindly without regard to the relevant context. All possible relevant senses of the multitude of words used in this disclosure may not be accounted for in a specific provided definition. The applicable sense or senses can depend on the specific context of the usage.

Initially, as a general aid to interpretation, the possible definitions of the words, phrases, and other symbols used herein are intended to be interpreted by reference to comprehensive general dictionaries of the English language published before or about the time of the earliest filing of this application for patent. A preferred dictionary is the American Heritage Dictionary of the English Language, 5^{th} Edition (Houghton Mifflin Harcourt, 2019). Where several different general definitions are available, it is intended that the broadest definitions or senses be selected that are consistent with this disclosure and the description of the presently most-preferred embodiments, including without limitation as shown in a Figure of any drawing.

After initially consulting such general dictionaries of the English language, it is intended that the words, phrases, or other symbols used herein be further interpreted or the most appropriate general definition or definitions be further selected by consulting technical dictionaries, encyclopedias, treatises, or relevant prior art to which the claimed invention pertains. If necessary to resolve any remaining doubt, utilizing the patent prosecution record may be helpful to select from among the possible interpretations.

Terms or phrases made up of more than one word (for example, compound terms or phrases or names) are sometimes not found in general dictionaries of the English language. Compound terms or names are to be interpreted as a whole, and not by parsing the separate words of the compound term, which might result in absurd and unintended interpretations. For example, in the context of railroad technology, a “coal car” does not mean a car made of coal but is well understood to mean the railroad car is for hauling coal. In general, compound terms are to be interpreted as they would be understood in the art and consistent with the usage in this specification.

Examining relevant general dictionaries, encyclopedias, treatises, prior art, and the patent record will make it possible to ascertain the appropriate meanings that would be attributed to the words and terms of the description and claims by those skilled in the art, and the intended full breadth of the words and terms will be more accurately determined. In addition, the improper importation of unintended limitations from the written description into the claims will be more easily avoided.

If there is any conflict in the usages of a word or term in this disclosure and one or more patent(s) or other documents that are incorporated by reference, the definitions that are consistent with the original material of this disclosure should be adopted in interpreting the original material of this disclosure, and the definitions that are consistent with the document incorporated by reference should be adopted in interpreting the material from that document.

Words of language often have multiple different senses. The selection of the applicable sense is usually understood from the particular context in which the word is used. If a word is specifically defined herein in a particular sense that does not reasonably apply in the context of a particular instance of usage elsewhere in the disclosure or claims, an applicable sense definition should be applied, not an inapplicable definition for a different context of usage. If any explicit definition herein is plainly obnoxious both to every ordinary meaning and to every technical meaning in the art for a usage in a particular context, the explicit definition herein should be disregarded as an obvious and unintended error.

Terms such as “first,” “second,” “third,” etc. (adjective) may be assigned arbitrarily and are merely intended to differentiate between two or more components, parts, or steps that are otherwise similar or corresponding in nature, structure, function, or action. For example, the words “first” and “second” serve no other purpose and are not part of the name or description of the following name or descriptive terms. The mere use of the term “first” does not require there be any “second” similar or corresponding component, part, or step. Similarly, the mere use of the word “second” does not require there be any “first” or “third” similar or corresponding component, part, or step. Further, the mere use of the term “first” does not require the element or step be the very first in any sequence, but merely that it is at least one of the elements or steps. Similarly, the mere use of the terms “first” and “second” does not necessarily require any sequence. Accordingly, the mere use of such terms does not exclude intervening elements or steps between the “first” and “second” elements or steps, etc.

If there is a discrepancy between the written description and one or more figures of the drawing, a person of skill in the art would recognize that the drawing is essentially correct and a person of skill in the art can understand the disclosure from the figures of the drawing.

Algebraic variables and other scientific symbols or notations used herein are selected according to convention, or, if no convention, arbitrarily. For example, the algebraic variables “a” and “b” can be selected arbitrarily.

An element, part, component, or ingredient can have more than one characteristic and that it can be characterized or classified in different, independent respects.

The headings and subheadings used in herein are intended for convenient reference but are not intended to be limiting.

### Patent Terminology

A patent “claim” (noun) means either: (a) a statement of the subject matter for which legal protection is sought in an application for patent; or (b) a statement of the subject matter for which legal protection has been granted (that is, legally recognized) in an issued patent. A patent claim is distinguishable from other types of legal claims and distinguishable from non-legal claims, such as factual or medical claims.

“Disclosure” (noun) (of an application for patent) means the specification of the written description with any original claims and any drawings, as of the effective filing date of the subject matter of a claim. The purpose of the disclosure is to disclose, that is, to make known. The applicable national law may provide a more particular definition or requirements for a disclosure of a patent.

An “original claim” (noun phrase) of a patent is a claim that is filed at the time of filing an application. An original claim is also part of the original disclosure. For the purposes of disclosure, an original claim can be treated as disclosure; however, for the purposes of examination of patentability and interpretation of full scope of an issued patent, any claim should be interpreted as broadly as literally stated except for any obvious error or except as may be interpreted under the doctrine of equivalents. For the purposes of disclosure, all variations, combinations, and permutations in which one or more limitations, elements, clauses, and descriptive terms from one or more of the original claims is introduced into another original claim are part of the disclosure. For example, any original claim that is dependent on another original claim can be amended to include one or more limitations found in any other original claim that is dependent on the same original base claim.

“Invention” (noun) means: (1) the act or process of inventing; or (2) a new and useful technological idea, such as for an article, manufacture, composition, machine, device, method, or process, or any new and useful improvement thereof.

“Patent” (noun) means: (1) a grant made by a government that confers upon the creator (or assignee) of an invention the right to exclude others from making, using, offering to sell, selling, or importing that invention within the territory of the government for a limited period of time; (b) letters patent; or (c) an invention protected by such a patent. The applicable national law may provide more particular requirements. The applicable national law may provide a more particular identification of the patent rights.

“Specification” (noun) means a written description of the ideas in an application or patent. The applicable national law may provide a more particular definition or requirements for a specification.

### Transitional Terminology

The words “comprising,” “containing,” “including,” “having,” “characterized by,” and all grammatical variations thereof are intended to have an open, non-limiting meaning as to any unstated limitations.

In a claim, the transitional term “comprising,” is inclusive or open-ended and does not exclude additional, un-recited elements or method steps. “Comprising” in claim language means the specified elements are essential, but other elements can be added and still form a construct within the scope of the claim. For example, a composition comprising an ingredient does not exclude it from having additional ingredients, an apparatus comprising a part does not exclude it from having additional parts, and a method having a step does not exclude it having additional steps.

In a claim, the transitional phrase “consisting essentially of” and all grammatical variations thereof are intended to limit the scope of a claim to the specified materials or steps and those that do not materially affect the basic and novel characteristic(s) of the claimed invention. A “consisting essentially of” claim occupies a middle ground between fully open claims using a “comprising” format and closed claims that are written in a “consisting of” format.

In a claim, the transitional phrase “consisting of” excludes any element, step, or ingredient not specified in the claim. For example, “consisting of” is defined as closing the claim to the inclusion of materials other than those recited except for impurities ordinarily associated therewith. For another example, a claim for a bone repair kit “consisting of” certain chemicals in a claim was infringed by a bone repair kit including a spatula in addition to the claimed chemicals because the presence of the spatula was unrelated to the subject matter of the claimed invention.

The phrase “selected from the group consisting of” (which is a kind of “Markush” grouping) means a list of alternative species within a grouping, even if the list includes the word “and.” For example, “selected from the group consisting of: a, b, and c” means any one or more of “a, b, and c”.

For the purposes of disclosure, however, such transitional phrases additionally subsume and include a disclosure of any more limited meanings. For example, a disclosure using the word “comprising” or like open-ended terms herein is intended to support a claim using any of the transitional terms “comprising,” “consisting essentially of,” or “consisting of” Similarly, a disclosure using the phrase “consisting essentially of” is intended to support a claim using the narrower phrase “consisting of”

### Other Grammar

“Phrase” (noun) means a sequence of two or more words that have meaning, especially when forming part of a sentence. “Noun phrase” means a phrase formed by a noun and all its modifiers and determiners; broadly any syntactic element (such as a clause, clitic, pronoun, or zero element) with a noun's function (such as the subject of a verb or the object of a verb or preposition), for example, the phrase “coal car” for which the head is the noun “car.” A noun phrase can be replaced by a single pronoun without rendering the sentence grammatically unacceptable.

The indefinite articles “a” or “an” mean at least one of the noun or noun phrase that the article introduces.

The conjunction “and” (in the sense of a listing or grouping) is open to additional elements or steps unless the context otherwise requires.

“Or” (conjunction) means: (1) (a) indicating an alternative, usually only before the last term of a series: hot or cold; this, that, or the other; (b) indicating the second of two alternatives, the first being preceded by either or whether; or (2) indicating a synonymous or equivalent expression.

For the purposes of disclosure, conjunctions “or” (in the sense of an alternative) and “and” (in the sense of a listing or grouping) can be interpreted first as open and non-limiting to other or additional possibilities, and, interpreted second, as closed and limiting.

For the purposes of disclosure, where elements are presented as groups or lists, for example, in “Markush group” format, each and every possible subgrouping of the grouped or listed elements is also disclosed as if set forth in separate lists. For example, where a disclosed group of three elements is disclosed, any subgrouping of one or two of the three elements is disclosed. For the purposes of disclosure, in various embodiments exactly one member of a group is present in, employed in or otherwise relevant to a given product or process. In various embodiments one, more than one, or all of a group's members are present in, employed in, or otherwise relevant to a given product or process.

“N/A” means not applicable or not determined, depending on the context.

The phrase “one or more” of something means an alternative grouping of the something.

In a “positive” (inclusionary) statement of a patent claim, an alternative grouping is met if any one or more of the statements of the grouping are met.

In a “negative” (exclusionary) statement of a patent claim, however, an alternative grouping is met only if all the statements of the grouping are met.

### General Terminology

“Acceptable” (adjective) means adequate to satisfy a need, requirement, or standard, as in at least sufficient.

“Active” (adjective) means: (a) being in physical motion; or (b) functioning or capable of functioning.

“Alternative” (adjective) means allowing for a choice between two or more things from which the choice can be made.

“Apparatus” (noun) means an integrated group of materials or devices for a particular purpose.

“Capable” (adjective) means having capacity or ability.

“Capacity” (noun) means the ability or extent of an ability, for example to receive, hold, dissolve, or absorb something or means the maximum amount that can be contained, held, dissolved, or absorbed, depending on the context.

“Carrier” (noun) means a mechanism, device, or composition by which something is conveyed or conducted.

“Characteristic” (noun) means a feature that helps to identify, tell apart, or describe recognizably, for example, a distinguishing mark, trait, or property.

“Control” (noun) means a comparison for checking or verifying the results of a test or scientific experiment or means a mechanism or other input that controls the operation of a machine, computer device, or process.

“Control” (verb) means to adjust to a requirement.

“Drawing” (noun) means: (1) the act or an instance of drawing; or (2) (a) the art of representing objects or forms on a surface chiefly by means of lines; (b) a work produced by this art. In a patent, a “drawing” may comprise one or more figures.

“Element” (noun) means a fundamental, essential, or irreducible constituent of a composite entity.

“Embodiment” (noun) means a concrete or embodied form of an abstract concept.

“Especially” (adverb) means to an extent or degree deserving of special emphasis; particularly, but not necessarily so limited.

“Essentially” (adverb) means constituting or being part of the fundamental nature or essence of something.

“General” (noun) means: (1) affecting or characteristic of the majority of those involved; or (2) involving only the main feature or features rather than precise or particular details.

“Improvement” (noun) means: (1) (a) the act or process of improving; or (b) the state of being improved; or (2) a change or addition that improves.

“Method” (noun) means a manner or way of doing something, especially a structured or systematic way of accomplishing something.

“Operative” (adjective) means: (1) functioning effectively; or (2) engaged in or concerned with physical, mechanical, electrical, or other activity.

“Process” (noun) means: (1) a series of actions, changes, or functions bringing about a result; (2) a series of operations performed in the making or treatment of a product, for example, a manufacturing process; or (3) a process, art, or method, and includes a new use of a known process, machine, manufacture, composition of matter, or material.

“Provide” (verb) means to furnish, supply, make available, or prepare. It can include making available to oneself. It does not require, but can include two or more individuals or actors, that is, it can include, but does not require a provider and a recipient.

“Select” (verb) means to choose from two or more alternatives.

“Significant” (adjective) means relatively large in importance, value, degree, amount, or extent in the relevant context. “Insignificant” means the opposite.

“Step” (noun) means one of a series of actions, processes, or measures taken to achieve a goal or purpose.

“Substantial” (adjective) and “substantially” (adverb) mean considerable in importance, value, degree, amount, or extent in the relevant context. “Substantial” is more, as a matter of degree, than “significant.”

“System” (noun) means a group of interacting, interrelated, or interdependent elements forming a complex whole.

“Use” (verb) means to put into service; to make work or employ something for a particular purpose or for its inherent or natural purpose.

“Usage” (noun) means the act of using, including usage data such as start time, end time, duration, type of activity, and intensity.

“Various” (adjective) means of diverse kinds purposefully arranged or grouped but not requiring uniformity.

“In various embodiments” (phrase) means one or more of various embodiments have the step, element, or attribute, but not all necessarily have it. Any of the various embodiments can be combined with any other of the various embodiments insofar as can be practical and non-contradictory to each other.

### Physical Conditions, Properties, and Phases

“Absence” (noun) means the state of being absent or only present at a concentration below the sensitivity of a test.

“Amount” (noun) means a number or a quantity (as of a measurement, such as of a mass, weight, volume, or concentration).

“Concentration” (noun) means the amount of a specified substance in a unit amount of another substance.

“Continuous phase” (noun) means the most external phase of a substance as a whole, regardless of the number of different internal phases or nested phases. Regarding a dispersion, the phase of the continuous phase is considered to be the phase of the substance as a whole, for example, a dispersion of solid particles suspended in a continuous liquid phase is considered to be a liquid, as a whole, and a dispersion of gas particles suspending in a continuous liquid phase is also considered to be a liquid, as a whole.

“Disperse” (verb) means to distribute (particles) evenly throughout a medium. “Dispersion” (noun) is a system in which particles of a substance of one chemical composition and physical state are dispersed in another substance of a different chemical composition or physical state. A dispersion can be classified in different ways, including, for example, based on the size of the dispersed particles, the uniformity or lack of uniformity of the dispersion, and, if a fluid, by whether or not precipitation occurs.

“Fluid” (noun) means a substance in a liquid or gaseous state. “Fluid” (adjective) means a state of matter in a liquid or gaseous state, at least regarding the continuous phase regarding a heterogeneous mixture.

“Foam” (noun) means a colloidal dispersion of a gas in a liquid or solid medium, such as shaving cream, foam rubber, or a substance used to fight fires. Typically, the volume of gas is much larger than that of the liquid or solid, with thin films separating gas pockets.

“Gas” (noun) means a substance in the gaseous state.

“Gaseous” (adjective) means the state of matter distinguished from the solid, gel, and liquid states by relatively low density, relatively low viscosity (i.e., much lower than that of water under standard laboratory conditions), relatively great expansion and contraction with changes in pressure and temperature, and the spontaneous tendency to diffuse and become distributed uniformly throughout any container or vessel.

“Liquid” (noun) means, depending on the context: (1) a substance that is liquid (adjective) at room temperature and pressure; (2) the state in which a substance exhibits a characteristic readiness to flow with little or no tendency to disperse and relatively high incompressibility (and not boiling, precipitating, or crystalizing); or (3) a substance in the fluid state of matter having no fixed shape but a fixed volume.

“Liquid” (adjective) regarding a substance means existing as or having characteristics of a liquid; especially tending to flow as a liquid.

“Material” (noun) means the tangible substance that goes into the makeup of a physical object, which can be constituted of one or more phases.

“Phase” (noun) means a substance having a chemical composition and physical state that is distinguishable from an adjacent phase of a substance having a different chemical composition or a different physical state.

“Pressure” (noun) in physics means force applied uniformly over a surface, measured as force per unit area.

“Room temperature” (noun phrase) means standard laboratory temperature.

“Solution” (noun) means a homogeneous mixture of two or more substances; frequently (but not necessarily) a liquid solution. A solution is a special type of homogeneous mixture. A solution is considered homogeneous: (a) because the ratio of solute to solvent is the same throughout the solution; and (b) because the solute will never settle out of the solution (in a liquid state), even under powerful centrifugation, which is due to intermolecular attraction between the solvent and the solute. An aqueous solution, for example, saltwater, is a homogenous solution in which water is the solvent and salt is the solute.

“Standard Laboratory Conditions” (noun phrase) means at a temperature of 77° F. (25° C.), at a pressure of 1 (one) atmosphere (101.325 kPa or 760 mmHg), without applied shear (e.g., any mixing force), and ambient relative humidity in the range of 40-60%.

“Standard laboratory pressure” or “standard pressure” or “normal pressure” (noun phrase) means 1 (one) atmosphere (101,325 Pascal).

“Standard laboratory temperature” or “normal temperature” (noun phrase) means at a temperature of 77° F. (25° C.).

“State” (noun) means a condition or mode of being, as with regard to circumstances. In chemistry and physics, it means the condition of a physical system with regard to phase, form, composition, or structure. The common physical states of matter include solid, liquid, and gas. Distinctions among these physical states are based on differences in intermolecular attractions. Solid is the state in which intermolecular attractions keep the molecules in fixed spatial relationships. Liquid is the state in which intermolecular attractions keep molecules in proximity (low tendency to disperse), but do not keep the molecules in fixed relationships. Gas is that state in which the molecules are comparatively separated, and intermolecular attractions have relatively little effect on their respective motions (high tendency to disperse). The physical state of a substance depends on temperature and pressure. If not other otherwise specifically stated, the physical state or phase or condition of a substance (or mixture of substances) and other physical properties are determined under Standard Laboratory Conditions.

“Substance” (noun) means that which has mass and occupies space, that is, matter; or a material of a particular kind or constitution.

“Stable” (verb) means resistant to change of position or condition, and in chemistry means not easily or rapidly decomposed or otherwise modified chemically. “Stability” (noun) means the state or quality of being stable, especially, resistance to change, displacement, or deterioration, and in chemistry meaning the state or quality of being stable chemically.

“Stabilize” (verb) means to make more stable, e.g., to increase to stability of something.

“Weight” (noun) means a measure of the heaviness of an object, and more particularly, the force with which a body is attracted to Earth or another celestial body, equal to the product of the object's mass and the acceleration of gravity. In the context of earth's gravitational constant, the weight of an object may be loosely equated to the mass of the object. For example, 2.2 pounds weight is equivalent to 1 kg mass, and grams or kilograms can be referred to as weights.

“By weight” (phrase) means according to weight rather than volume or other measure. A concentration by weight can be expressed as a “weight percent” or as a proportion “weight/weight” or abbreviated to “w/w”. For example, “by weight” or “w/w” means by weight of the composition.

### Oil and Gas Field Terminology

“Aqueous” means relating to, similar to, containing, or dissolved in water.

“Aqueous phase” (noun phrase) means a liquid where the medium phase is of water.

“API” means the American Petroleum Institute.

“API gravity” means the API measure of how heavy or light a petroleum liquid is compared to water, often expressed as “degrees” of API gravity. If the API gravity is greater than 10, it is lighter and floats on water; if the API gravity is less than 10, it is heavier and sinks.

“Brine” means an aqueous solution containing a sufficient concentration of one or more dissolved inorganic salts to cause the aqueous solution to have a higher density than typical seawater. Classes of brines include those of chlorides, bromides, or formates.

“Chemical” (noun) means a substance with a distinct molecular composition. A pure chemical is a sample of matter that cannot be separated into simpler components without chemical change.

“Chemical” (adjective) means of or relating to chemistry or means of or relating to the properties or actions of chemicals, depending on the context. “Chemically” (adverb) means of or relating to chemistry or means of or relating to the properties or actions of chemicals, depending on the context.

“Condensate” (noun) means a low-density, high-API gravity liquid hydrocarbon that often occurs in association with natural gas. The API gravity of condensate is typically about 50 degrees to about 120 degrees. Condensate is formed through retrograde condensation. The formation of condensate depends on temperature and pressure conditions allowing for condensation of liquid from gas falling below the dewpoint. During production, there is a risk of the hydrocarbon changing from gas to liquid if the reservoir pressure drops below the dewpoint. Reservoir pressure can be maintained by fluid injection if gas production is preferable to liquid production. Gas produced in association with condensate is called wet gas.

“Condensate liquid” (noun) means hydrocarbons that are in the gaseous phase at reservoir conditions of temperature and pressure but condense into the liquid phase as they move up a wellbore.

“Dewpoint” (noun) means the pressure at which a condensate liquid comes out of solution in a gas phase.

“Emulsion” (noun) means a mixture of dispersed-phase liquid droplets in an external phase that is another liquid.

“Foamer” (noun) also known as “foaming agent” (noun phrase) or “foaming surfactant” (noun phrase) is a type of surfactant that induces or increases the formation of foam of a gas and a liquid.

“Hydrocarbon” (noun) means a naturally occurring organic chemical including hydrogen and carbon atoms. A hydrocarbon can be methane (that is, CH₄) or a more complex chemical of hydrogen and carbon atoms.

“Produced water” (noun phrase) means water produced at the surface from a wellbore, where the water is not a treatment fluid. The characteristics of produced water can vary, and the term often implies an inexact or unknown composition.

“Retrograde condensation” (verb phrase) means the formation of liquid hydrocarbons in a gas reservoir as the pressure in the reservoir decreases below dewpoint pressure during production. It is called “retrograde” because some of the gas condenses into a liquid under isothermal conditions instead of expanding or vaporizing when pressure is decreased.

“Water” (noun) means pure water, or, more broadly, of water, such as a brine or produced water, or having a continuous phase of water, depending on the context.

### CONCLUSION

Therefore, the disclosure can be understood by a person of skill in the art to obtain the purposes and advantages mentioned as well as those that are inherent therein.

The various disclosed embodiments are illustrative only, as the disclosure can be modified and practiced in different but equivalent manners apparent to those skilled in the art having the benefit of the teachings herein. It is, therefore, evident that the particular illustrative embodiments disclosed above can be altered or modified and all such variations are considered within the scope of the disclosure.

The various elements or steps according to the disclosed elements or steps can be combined advantageously or practiced together in various combinations or subcombinations of elements or sequences of steps to increase the efficiency and benefits that can be obtained from the disclosure.

One or more of the above and various embodiments can be combined with one or more of the other various embodiments, unless explicitly stated otherwise.

The illustrative disclosure can be practiced in the absence of any element or step that is not specifically disclosed or claimed.

Any embodiment of the disclosure that falls within the prior art can be explicitly excluded from any one or more of the claims. Because such embodiments are deemed to be known to one of ordinary skill in the art, they can be excluded even if the exclusion is not set forth explicitly herein.

Any embodiment of the disclosure can be explicitly excluded from a particular patent claim, for any reason, whether or not related to the existence of prior art. Where elements are presented as lists, for example, in Markush group format, each subgroup of the elements is also disclosed, and any element or elements can be removed from the claimed group.

Those of ordinary skill in the art will recognize or be able to ascertain using no more than routine experimentation many equivalents to the specific embodiments of this disclosure. Those of ordinary skill in the art will appreciate that various changes and modifications to this description can be made without departing from the spirit or scope of the disclosure.

The description of the specific examples herein does not necessarily point out what an infringement would be but are to provide at least one explanation of how to make and use the disclosure.

Furthermore, no limitations are intended to the details of composition, design, construction, or steps of the disclosure, other than as set forth in a specific claim.

What is claimed:

1. A system for evaluating a foamer for unloading liquid, the system comprising:
(a) a temperature-controlled bath having a first vessel for containing a liquid in the first vessel at a temperature controlled by the temperature-controlled bath and for measuring the volume of a liquid or foam in the first vessel;
(b) a gas tubing operatively connected from a gas flow meter and a gas source to gas delivery tubing and a frit for sparging the liquid in the first vessel with a gas for making a foam;
(c) a mass balance;
(d) a second vessel on the mass balance; and
(e) fluid tubing operatively connected from the first vessel in the temperature-controlled bath to the second vessel.

2. The system according to claim 1, additionally comprising a condenser for the fluid tubing.

3. The system according to claim 1, additionally comprising:
(a) a camera for recording changes in visual appearance of the foam in the first vessel of the temperature-controlled bath;
(b) a central processing unit operatively connected to the mass balance and to the camera for recording and analyzing data from the mass balance and from the camera; and
(c) a user interface operatively connected to the central processing unit.

4. A method for evaluating a foamer, the method comprising the steps of:
(a) combining (i) an aqueous phase, a hydrocarbon phase, or both an aqueous phase and a hydrocarbon phase in a predetermined proportion with (ii) a foamer to obtain a liquid, wherein the foamer is in a predetermined concentration in the liquid;
(b) sparging the liquid with a gas under sparging conditions including a predetermined gas flow rate to create a foam from at least some of the liquid and at least some of the gas; and
(c) during or after the step of sparging, determining the amount of the liquid in the foam, wherein the step of determining is performed one or more times.

5. The method according to claim 4, wherein the aqueous phase is water, a synthetic water composition simulating water obtained from a well, or a sample of water obtained from a well.

6. The method according to claim 4, wherein the hydrocarbon phase is kerosene, a hydrocarbon composition simulating a hydrocarbon obtained from a well, or a sample of a hydrocarbon obtained from a well.

7. The method according to claim 4, wherein the foamer is a hydrophilic foamer or an amphiphile foamer.

8. The method according to claim 4, wherein the gas is selected from the group consisting of air, nitrogen, carbon dioxide, or any combination thereof in any proportion.

9. The method according to claim 4, wherein the step of sparging additionally comprises controlling the temperature of the liquid during the step of sparging.

10. The method according to claim 4, wherein the step of determining the amount of the liquid in the foam comprises the steps:
(a) measuring the volume of the liquid before the step of sparging;
(b) during or after the step of sparging, measuring the volume of the liquid remaining that is not in the foam; and
(c) subtracting the volume of liquid remaining that is not in the foam from the volume of the liquid before the step of sparging to determine a difference that is the amount of the liquid in the foam.

11. The method according to claim 4, wherein the step of determining the amount of the liquid in the foam comprises the steps:
(a) recording the volume of the liquid before the step of sparging using a camera operatively connected to a central processing unit;
(b) over time during or after the step of sparging, recording the volume of the liquid remaining that is not in the foam using the camera operatively connected to a central processing unit; and
(c) analyzing the recorded volumes over time using the central processing unit to determine the differences that are the volumes of the liquid in the foam over time.

12. The method according to claim 11, wherein recording the volume of the liquid is with a graduated cylinder having graduated markings.

13. The method according to claim 11, additionally comprising the step of graphically plotting the recorded volumes over time using the central processing unit operatively connected to a user interface.

14. The method according to claim 4, additionally comprising the step of, over time during or after the step of sparging, recording with a camera the visual appearance of the foam as it changes over time.

15. The method according to claim 4, wherein the step of determining the mass or the amount of the liquid in the foam comprises the steps of:
(a) collecting the foam to obtain a collected foam and any of the liquid dropped from the collected foam; and
(b) measuring the mass of the collected foam and any of the liquid dropped from the collected foam.

16. The method according to claim 15, wherein the step of collecting the foam additionally comprises cooling the foam to reduce any evaporation of the liquid in the foam to the atmosphere.

17. The method according to claim 16, wherein cooling the foam is with a condenser.

18. The method according to claim 16, additionally comprising the steps of:
(a) over time during or after the step of sparging, recording the mass of the collected foam and any of the liquid dropped from the collected foam; and
(b) analyzing the recorded masses using a central processing unit to determine the amounts of the liquid in the collected foam over time.

19. The method according to claim 4, additionally comprising the steps of:
(a) during or after the step of sparging, determining the volume of the foam; and
(b) based on the step of determining the amount of the liquid in the foam, determining the density of the foam.

20. The method according to claim 4, wherein the steps of combining, sparging, and determining the amount of the liquid in the foam are steps of a discrete and separate test for the liquid and for the sparging conditions from another discrete and separate test for a different liquid or for different sparging conditions.
